SEQUENCE LISTING (7 sequences)

SEQ ID NO: 1 - DNA, 718 bp, Zea mays
  source 1..718: mol_type = genomic DNA
gcggtttcgttctccttcctgccgctggccggccggctggtggcaagctcgatcgagctaggagtcagatggaaggcacgatgaagcacagcaacgttgtgacggagaggagctctgtgaccaagccccgtgaacaggaggtgtccgcttggcatgatgaggctggagagcgtctggtttggacgccgagagcagcagttcagctgatgcttcatgcctagcatcagcacggcgttatcctaattgagcttttcagccaccatctttatccttcctcatattctccatatcgtgatgcttgtggtttcgcaagatcaggtcttcttgaccttgcaagaccttttcttgaccttgtaagacccaactctacgatacatcttttctttgacctaatcttctctacctcacacctttctatctgttcttacttctgttctatgtctatcccttctgagctgtgttgagctactttgataaagctaggtactatagattaagtgttttaggaggtgtttctcacatgtgtaatgttgtgagcttgccgtgtgggaaaaaaaaaatccattttgatggtagctatatagagtgaggcatgagacaaacaaaagtatgttcctgttgcgccatgagcacatgatgctcggccatgtgaagtaggcgcctacatgcaagctaagctagctagctccgtatgtatcgcctcgatggaataagctgc

SEQ ID NO: 2 - DNA, 41 bp, synthetic construct
  source 1..41: mol_type = other DNA
gggggtaccggatccgaattcgcggtttcgttctccttcct

SEQ ID NO: 3 - DNA, 41 bp, synthetic construct
  source 1..41: mol_type = other DNA
agagttgttgattcagaattcgcagcttattccatcgaggc

SEQ ID NO: 4 - DNA, 33 bp, synthetic construct
  source 1..33: mol_type = other DNA
aatttcctattgcgggtgctgatgctaggcatg

SEQ ID NO: 5 - DNA, 32 bp, synthetic construct
  source 1..32: mol_type = other DNA
ccgcaataggaaattgagcttttcagccacca

SEQ ID NO: 6 - DNA, 44 bp, synthetic construct
  source 1..44: mol_type = other DNA
gcaggtcgactctagaggatccgcggtttcgttctccttcctgc

SEQ ID NO: 7 - DNA, 46 bp, synthetic construct
  source 1..46: mol_type = other DNA
tcgagctcggtacccggggatccgcagcagcttattccatcgaggc

(19) United States
(12) Patent Application Publication
(10) Pub. No.: US 2025/0163441 A1
(43) Pub. Date: May 22, 2025

(54) Maize ZmTAS3j Gene and Its Use in Improving Tolerance to Lead for Plant

(71) Applicant: SICHUAN AGRICULTURAL UNIVERSITY, Chengdu, Sichuan (CN)

(72) Inventors: Yaou Shen, Chengdu (CN); Zhaoling Li, Chengdu (CN); Langlang Ma, Chengdu (CN); Chaoying Zou, Chengdu (CN); Peng Liu, Chengdu (CN); Zhong Chen, Chengdu (CN); Guangsheng Yuan, Chengdu (CN); Guangtang Pan, Chengdu (CN)

(21) Appl. No.: 19/026,817

(22) Filed: Jan. 17, 2025

Related U.S. Application Data
(63) Continuation of application No. PCT/CN2023/134244, filed on Nov. 27, 2023.

(30) Foreign Application Priority Data
Nov. 17, 2023 (CN) 202311537245.3

(51) Int. Cl. C12N 15/82; C07K 14/415

(52) U.S. Cl. CPC C12N 15/8205; C07K 14/415; C12N 15/8213; C12N 15/8271

(57) ABSTRACT

The present disclosure relates to the ZmTAS3j mediation lead stress tolerance in maize. The sequence of ZmTAS3j is identified by SEQ ID NO:1. The invention cloned the gene of ZmTAS3j from maize, and through genetic transformation of Arabidopsis thaliana and maize, it was verified that the ZmTAS3j mediated lead stress tolerance in maize.

### CROSS-REFERENCE TO RELATED APPLICATIONS

The present application is a Continuation Application of PCT Application No.PCT/CN2023/134244, filed on Nov. 27, 2023, which claims the priority of Chinese Utility Model Application No. 202311537245.3, filed on Nov. 17, 2023, the entire contents of which are hereby incorporated by reference.

### REFERENCE TO AN ELECTRONIC SEQUENCE LISTING

The contents of the electronic sequence listing (Maize ZmTAS3j Gene and Its Use in Improving Tolerance to Lead for Plant.xml; Size:7,761 bytes; and Date of Creation: Nov. 17, 2023) is herein incorporated by reference in its entirely.

### TECHNICAL FIELD

The present invention relates to the technical field of genetic engineering, and in particular to the Maize ZmTAS3j gene and it use in improving tolerance to lead for plant.

### DESCRIPTION OF THE PRIOR ART

Maize (Zea mays L.) is an important cereal crop cultivated under various environmental conditions. Soil contamination by heavy metals is a major environmental stress that has detrimental effects on most crops. Lead (Pb) is a prominent environmental contaminant that inhibits key enzyme activities, increases the generation of reactive oxygen species (ROS), induces DNA damage, generates lipid peroxidation, causes protein oxidation, and ultimately promotes cell death in plants. These effects are particularly harmful to maize biomass and grain yield. Furthermore, Pb can enter the human body through the food chain, water, and air, accumulating over time and posing serious threats to human health. Therefore, an in-depth study of the molecular mechanisms underlying Pb resistance in maize, the identification of new Pb-resistant genes, and the cultivation of maize germplasm with high or low Pb accumulation are crucial strategies for remediating Pb-contaminated soil or reducing Pb accumulation in maize. Small RNAs play an important role in plant responses to biotic stress, as well as in leaf and root development and abiotic stress. Among these, phasiRNA is a type of small RNA produced by the cleavage of RNA transcripts mediated by miRNA, functioning similarly to miRNA. It directly targets and cleaves endogenous complementary mRNA, thereby achieving post-transcriptional gene silencing (PTGS). This regulatory mechanism is believed to have evolved in plants and is involved in the response to various biotic and abiotic stresses. However, it remains unreported whether phasiRNA is involved in the response to Pb stress during the seedling stage of maize.

PhasiRNA refers to endogenous siRNA with lengths of 21 nt or 24 nt that are arranged head-to-tail. The primary phasiRNA, produced through the targeted cleavage of transcripts by miRNA, is processed by RDR6 in conjunction with the RNA-binding protein SGS3, resulting in the synthesis of double-stranded RNA that forms pre-phasiRNA. This pre-phasiRNA is subsequently transported into the nucleus, where, with the assistance of double-stranded RNA binding protein 4 (DRB4), it is cleaved by Dicer-like enzymes (DCLs) to a fixed length, yielding phasiRNA that is also connected head-to-tail and exhibits a phase arrangement. Notably, the phasiRNA produced by DCL4 cleavage is 21 nt in length. In instances where DCL4 is mutated, DCL2 and DCL3 can compensate for its function. Conversely, the phasiRNA generated by DCL5 (DCL3b) cleavage is 24 nt in length and is predominantly found in the regenerative tissues of monocots. Additionally, 24 nt PHAS sites have also been identified in dicotyledons. These sites capable of producing phasiRNA are referred to as ‘PHAS’ sites. Trans-acting siRNA (tasiRNA) is a subtype of phasiRNA, originating from non-coding RNA and representing a plant-specific endogenous small RNA. The site of production for this type of small RNA is designated as the “TAS” site. In Arabidopsis thaliana, only eight TAS sites have been documented: TAS1a/b/c, TAS2, TAS3a/b/c, and TAS4. Among these, TAS1a/b/c and TAS2 are targeted and recognized by miR173, while TAS3a/b/c and TAS4 are recognized by miR390 and miR828, respectively. Notably, TAS3 is regarded as a conserved TAS site. In addition to targeting the ARF family, tasiRNA derived from TAS3 may have other potential targets, suggesting that tasiRNA and its complex regulatory network could play a significant role in plant stress adaptation.

### SUMMARY OF THE DISCLOSURE

In order to solve the above technical problems, the present disclosure provides the Maize ZmTAS3j gene and it use in improving tolerance to lead for plant. The identification and functional analysis of PHAS gene and phasiRNA that respond to Pb stress in maize seedling stage according to the present disclosure will provide a new perspective for key genes and their expression regulation networks under Pb stress in plants.

Technical scheme of the invention are the Maize ZmTAS3j gene and it use in improving tolerance to lead for plant.

The nucleotide sequence of ZmTAS3j is SEQ ID NO:1.

Overexpression of ZmTAS3j in plant was a method to improve plant tolerance to Pb stress.

A method of ZmTAS3j-overexpression in Arabidopsis thaliana was as follows:

The total RNA of B73 inbred lines was extracted and reverse-transcribed into cDNA. The sequences of ZmTAS3j were amplified, and constructed into pRI101-AN expression vector with EcoRI as the restriction site. The vector was named AN101-ZmTAS3j (FIG. 12A).

(2) The 5′ target of miR390 in the sequence of ZmTAS3j was mutated by mismatch PCR amplification, and the 5′ target of miR390 was destroyed. The mutant sequence of ZmTAS3j was constructed into pRI101-AN expression vector with EcoRI as the restriction site. The vector was named AN101-ZmTAS3jmut (FIG. 12B).

(3) The plasmid AN101-ZmTAS3j and AN101-ZmTAS3jmut were transformed into Agrobacterium GV3101 respectively. Arabidopsis transgenic plants were obtained by inflorescence soaking.

The method described in claim 4 is characterized in that the homologous recombination primer of the step (1), the nucleotide sequence of AN101-ZmTAS3j-F, such as SEQ ID NO:2, the nucleotide sequence of AN101-ZmTAS3j-R, such as SEQ ID NO:3.

The method described in claim 4 is characterized in that the homologous recombination primer of the step (2), the nucleotide sequence of ZmTAS3jmut-FL-R, such as SEQ ID NO:4, the nucleotide sequence of ZmTAS3jmut-FL-F, such as SEQ ID NO:5.

The overexpression methods of ZmTAS3j in maize were characterized as follows: with Ubi as the promoter, nos as the terminator, bar as the selective marker gene. According the sequence of ZmTAS3j, amplification primers were designed, and the ZmTAS3j was amplified. The overexpression vector of OE-ZmTAS3j was constructed by homologous recombination using BamHI as the restriction site.

Among the above methods, the primer used in constructing OE-ZmTAS3j vector by homologous recombination method as follow: OE-ZmTAS3j-F, SEQ ID NO:6, OE-ZmTAS3j-R, SEQ ID NO:7.

To investigate the impact of phasiRNA on lead tolerance in maize, this study constructed 305 small RNA databases from a genetically diverse population of maize inbred line. Through bioinformatics analysis, a total of 41 phasiRNAs derived from 9 PHAS genes were identified, revealing a significant negative correlation with lead (Pb) content in maize roots. Expression genome-wide association analysis (eGWAS) indicated that only the expression of PHAS_1 is regulated by its cis-expression quantitative trait locus (cis eQTL) and exhibits the highest phase score. The phasiRNA derived from this gene, named ZmTAS3j, also demonstrated a significant negative correlation with Pb content in maize roots. This study represents the first successful cloning of the ZmTAS3j gene from maize, and subsequent genetic transformation into Arabidopsis thaliana and maize (Zea mays) showed that overexpression of the ZmTAS3j gene enhances plant tolerance to Pb stress. Inductively coupled plasma mass spectrometry, transmission electron microscopy, and EDS X-ray microanalysis were employed to quantify lead content in the roots of both overexpression (OE) and wild type (WT) strains under control (CK) and Pb treatment conditions. By observing the deposition sites of Pb aggregates and analyzing the deposits in root tissues, the results indicated that ZmTAS3j negatively regulates cell wall thickness in maize root cells and the accumulation of Pb polymers, thereby influencing Pb content in maize roots. Finally, enzyme-linked immunosorbent assay (ELISA) detection kits and various other assays were utilized to measure IAA levels, as well as the activities of superoxide dismutase (SOD), peroxidase (POD), catalase (CAT), and the levels of hydrogen peroxide (H2O2) and malondialdehyde (MDA) in the roots of both OE and WT strains under CK and Pb treatment conditions. The results demonstrated that, compared to WT roots, the levels of IAA were elevated, while the activities of SOD and CAT enzyme activities increased, and the levels of H2O2 and MDA decreased in OE roots. In summary, the overexpression of tasiRNA derived from ZmTAS3j may enhance IAA levels in maize roots by regulating the auxin anabolic pathway and the expression of ROS-responsive genes. This process promotes the growth of primary roots and strengthens the activity of ROS scavenging enzymes, thereby facilitating the removal of ROS in the body and reducing the associated damage to plants.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1A illustrates construction of small RNA library.

FIG. 1B illustrates identification of PHAS loci.

FIG. 2A shows abundance distribution of 20-24 nt small RNAs.

FIG. 2B shows abundance distribution of 20-21 nt small RNAs.

FIG. 3A to FIG. 3D show Clustering and heat map of phasiRNA and GO enrichment of predicted target genes, wherein FIG. 3A shows K-mean clustering based on the expression of 55 phasiRNAs; FIG. 3B shows significance of phasiRNA expression among groups; FIG. 3C shows significance of Pb content among groups; and FIG. 3D shows GO enrichment of phasiRNA target genes. RLC, root lead content; SLC, shoot lead content.

FIGS. 4A-4G show Manhattan plots for expression-based genome-wide association studies (eGWAS) of the four PHAS genes negatively correlating with Pb content in 305 maize lines, wherein FIGS. 4A, 4B, 4C, and 4D represent PHAS1, PHAS_2, PHAS_6, and PHAS_12, respectively, the dashed red lines denote the threshold of P value (1.14×10⁻⁶). PHAS, genes producing phasiRNAs; FIG. 4E shows expression levels of PHAS_1 in two haplotypes (GG and AA) divided by the lead SNP PZE-109085594, TPM, transcripts per million; FIG. 4F shows difference in Pb content performance between different haplotypes; and FIG. 4G shows genomic configuration of ZmTAS3j their read abundance distribution and sequence alignment in SCL114 and SCL312 (haplotype: GG) and SCL011 and SCL064 (haplotype: AA) viewed together with 21 nt phasiRNAs of ZmTAS3j in IGV.

FIG. 5 shows map of the vector 35S: GFP-ZmTAS3j.

FIG. 6A shows a vector map of AN101-MIR390.

FIG. 6B shows a vector map of STTM390.

FIGS. 7A-7F illustrate predicted foldbacks of MIR390, MIRD5(+) mutant and MIRD6(+); where FIGS. 7A and 7B, miR390; FIGS. 7C and 7D, miRD5(+); FIGS. 7E and 7F, miRD6(+).

FIGS. 8A-8C show miR390 directly cleaving the mRNA of ZmTAS3j; wherein FIG. 8A shows transient expression of green fluorescence in tobacco leaves; FIG. 8B. shows expression level of ZmTAS3j in tobacco leaves; and FIG. 8C. shows expression level of eGFP in tobacco leaves.

FIGS. 9A-9C show the miR390-guided target cleavage was further confirmed for ZmTAS3j by 5′RACE, wherein FIG. 9A shows a specific product of nested PCR amplification, “out” refers to the specific product of first round, and “inner” refers to the specific produce of second round; FIG. 9B shows distribution ratio of sequencing results, the numbers above the vertical arrows indicate the data from 5′RACE confirmation; and FIG. 9C shows predicted target site of miR390 were consistent with 5′RACE sequencing results.

FIGS. 10A-10C show organization of tasiRNA constructs and expression of tasiRNAs derived from ZmTAS3j; wherein FIG. 10A shows organization of tasiRNA constructs, D1(+) to D10(+) represent the positions of tasiRNA derived from the 3′ to 5′direction, D1(−) to D10(−) represent the positions of tasiRNAs derived from the 5′ to 3′ direction; FIG. 10B shows expression levels of tasiRNA from the 3′ to 5′ direction; and FIG. 10C shows expression of tasiRNA from the 5 to 3 directions.

FIGS. 11A and 11B show tasiD5(+) and tasiD6(+) directly cleaved the mRNA of ZmARF3; wherein FIG. 11A shows Green fluorescence decreased with tasiD5(+) or tasiD6(+); and FIG. 11B shows mRNA of ZmTAS3j decreased with tasiD5(+) or tasiD6(+).

FIG. 12A shows vector map of AN101-ZmTAS3j.

FIG. 12B shows vector map of AN101-ZmTAS3jmut.

FIGS. 13A and 13B show Phenotypes of WT, OE-ZmTAS3j and OE-ZmTAS3jmut; wherein FIG. 13A shows Phenotypes of WT, ZmTAS3j-OE, and ZmTAS3jmut-OE lines under ½ MS medium for 10 days; and FIG. 13B shows Phenotypes of WT, ZmTAS3j-OE, and ZmTAS3jmut-OE lines under ½ MS medium with 200 mg/L Pb(NO3)2 after 10 days.

FIGS. 14A and 14B show Statistical analysis of the phenotypic data in WT, OE-ZmTAS3j and OE-ZmTAS3jmut lines; wherein FIG. 14A shows statistical analysis of rosette, root length and plant height; and FIG. 14B shows number of lateral roots.

FIG. 15 shows map of OE-ZmTAS3j vectors.

FIGS. 16A to 16I show phenotype comparison between ZmTAS3j-overexpression and wild-type lines in maize, seedlings grown aerobically for 7 days were divided into two groups, and transferred to control and Pb treatment conditions and further grown for 7 days; wherein FIG. 16A shows model of ZmTAS3j-overexpression vector, the blue arrow represents the promoter of ZmTAS3j, the gray rectangles represent the exons of ZmTAS3j gene, And the red rectangles represent the miR390 cleavage site; FIG. 16B shows analysis of ZmTAS3j expression in WT, OE-1, and OE-2; FIG. 16C shows analysis of tasiARFs (D5+) and (D6+)] expression in WT, OE-1, and OE-2; FIG. 16D shows expression analysis of ZmARF3 in control and Pb treatment, after 7 days of growth under control and Pb treatment conditions, Primary root lengths (PRL); FIG. 16E shows primary root lengths (PRL); FIG. 16F shows root dry weights (RDW); FIG. 16G shows total root lengths (TRL); FIG. 16H shows pictures of wild type (WT) and ZmTAS3j-overexpressed lines (OE) under control condition; and FIG. 16I shows pictures of wild type (WT) and ZmTAS3j-overexpressed lines (OE) after 7 days of Pb treatment, wherein scale bars=1 cm, data are presented as the mean±SD in panel FIGS. 16B, 16C, 16D, 16E, 16F, and 16G, and significance (P<0.05) was determined using Student's t-test [n=3 (FIGS. 16B, 16C, and 16D) and n >20 (FIGS. 16E, 16F, and 16G); OE, ZmTAS3j-overexpressed. WT, wild-type maize KN5585. CK, control conditions.

FIGS. 17A-17I show statistical analysis of Pb content and effects of Pb exposure on root tissue structure and ultrastructure in ZmTAS3j-overexpressed line; wherein FIG. 17A shows Pb content in leaf and root; FIG. 17B shows total Pb content in whole seedling; FIG. 17C shows total area of the peaks representing the Pb aggregates; FIGS. 17D and 17F show transmission electron microscope (TEM) images of root tissue of WT and OE lines after Pb treatment, respectively; FIGS. 17E and 17G show Pb aggregates in the cytoplasm of root tissues of WT and OE lines after Pb treatment, respectively; FIGS. 17H and 17I shows results of energy-dispersive X-ray spectroscopy (EDS) of root tissues of WT and OE lines after Pb treatment, respectively; and wherein CW, cell wall; CT, cytoplasm; IS, intercellular space; aggregates, Pb aggregates. OE, ZmTAS3j-overexpressed; WT, wild-type maize KN5585, the data are plotted as means±SD (n=3) in panel FIG. 17A, FIG. 17B, and FIG. 17C, and significance (P<0.05) was determined using Student's t-test.

FIGS. 18A-18N show indole acetic acid (IAA) levels and the contents of H2O2, malondialdehyde (MDA), superoxide dismutase (SOD), peroxidase (POD), and catalase (CAT) in WT and OE roots; wherein FIG. 18A shows IAA levels in the roots during 0-72 h under control conditions; FIG. 18B shows IAA levels in the roots during 0-72 h under Pb treatment; FIG. 18C and FIG. 18D show content of H₂O₂ in the roots during 0-72 h under control and Pb conditions, respectively; FIG. 18E and FIG. 18F show 3,3-diaminobenzidine (DAB) staining of leaves under control and Pb stress conditions, respectively, scale bars=1 cm; FIG. 18G and FIG. 18H show content of MDA in the roots during 0-72 h under control and Pb conditions, respectively; FIGS. 18I and J show content of SOD in the roots during 0-72 h under control and Pb stress conditions, respectively; FIG. 18K and FIG. 18L show content of POD in the roots during 0-72 h under control and Pb stress conditions, respectively; FIG. 18M and FIG. 18N show content of CAT in the roots during 0-72 h under control and Pb conditions, respectively; and wherein data are presented as the mean±SD (n=3), significant differences are indicated by the number of asterisks; one asterisk (*) denotes P<0.05, two asterisks (**) denote P<0.01, as determined by Student's t-test.

FIGS. 19A-19E show response of differentially expressed genes (DEGs) to Pb stress in the wild-type (WT) and overexpression (OE) lines of maize; wherein 19A is Venn diagram of DEGs showing differential responses to Pb stress between WT and OE lines; FIG. 19B shows that the number of the up- and down-regulated genes were showed in histogram; FIG. 19C shows Heatmap of gene expression in all experimental groups;

FIG. 19D shows Gene Ontology enrichment bar graph of DEGs specifically expressed in OE lines under Pb stress; and FIG. 19E. shows expression heatmap of the genes with specifically high expression in the OE line under Pb stress; and wherein red and blue colors in the heatmap represent the upregulated and downregulated genes, respectively.

### DESCRIPTION OF EMBODIMENTS

### Materials and Results

1. Identification of PHAS Sites and phasiRNA Responding to Pb Stress in Maize

### 1.1 Materials and Methods

1.1.1 The study utilized a total of 305 inbred maize (Zea mays L.) lines obtained from the Southwest China breeding program. The population materials were supplied by the Maize Research Institute of Sichuan Agricultural University, and primarily originated from China, the United States, and Mexico.

### 1.1.2 Methods

### (1) The Formula of Nutrient Solution for Maize Seedlings.

The nutrition solution for maize used in this experiment was improved by Hoagland formula, as shown in Table 1.

TABLE 1
The formula of nutrient solution for maize seedlings

| Ingredient | concentration | | g/mol | mg/L |
|---|---|---|---|---|
| KNO₃ | 5 | mmol/L | 101.1 | 506 |
| Ca(NO₃)₂•4H₂O | 4 | mmol/L | 236.5 | 945 |
| NH₄NO₃ | 1 | mmol/L | 80 | 80 |
| KH₂PO₄ | 1 | mmol/L | 136.09 | 136 |
| MgSO₄•7H₂O | 2 | mmol/L | 246.47 | 492.3 |
| FeNaEDTA | 10 | μmol/L | 367.47 | 36.7 |
| KI | 5 | μmol/L | 166 | 0.83 |
| H3BO3 | 0.1 | mmol/L | 61.83 | 6.2 |
| ZnSO4•7H2O | 30 | μmol/L | 287.56 | 8.6 |
| CuSO4•7H2O | 0.1 | μmol/L | 249.69 | 0.0249 |
| MnSO₄•H₂O | 0.1 | mmol/L | 169.01 | 16.9 |
| KH₂PO₄ | 1 | mmol/L | 115.03 | 115.03 |
| Na₂MoO₄•2H₂O | 1.2 | μmol/L | 205.92 | 0.25 |
| CoCl₂•6H₂O | 0.1 | μMol/L | 237.93 | 0.025 |

### (2) Maize Seedling Culture and Stress Treatment

1) From the same batch of seeds, select uniform and plump seeds for subsequent use.

2) Soak the seeds in a 10% hydrogen peroxide (H₂O₂) solution for 15 minutes, followed by three rinses with distilled water. Afterward, soak the seeds in saturated calcium sulfate (CaSO4) overnight.

3) Rinse the soaked seeds three times with distilled water, then transfer them to a germination box, arranging them neatly, and cultivate them in the dark at 28° C. for germination.

4) Once the seeds have germinated to approximately 1 cm, transfer them into vermiculite and place them in the artificial climate chamber at the Maize Research Institute, where the temperature and light-dark cycle are set to 28° C. for 14 hours and 22° C. for 10 hours, respectively, until the maize seedlings reach the three-leaf and one-heart stage.

5) Select thirty maize seedlings exhibiting consistent growth, remove the endosperm, and wash the remaining vermiculite from the roots using clean water. Fix the maize seedlings with foam rings and transfer them to a hydroponic device filled with a semi-nutrient solution, allowing them to adaptively cultivate for three days. Subsequently, transfer the maize seedlings into a nutrient solution containing 1 mM Pb(NO3)2 for stress treatment.

6) After 24 hours of Pb stress treatment, all treated seedlings were washed twice with EDTA solution and then twice with clean water. The sampling was divided into two parts: first, samples for sRNA sequencing were obtained by randomly selecting three plants from each inbred line and extracting roots for RNA extraction and sequencing. Second, samples for Pb content measurement were collected from each inbred line after stress treatment. The roots and aboveground parts of the cross lines were placed in kraft paper bags and dried in an oven at 80° C. until they reached a constant weight for subsequent Pb content determination.

(3) Extraction and Detection of RNA from Root Tissue of Maize Seedling

1) Total RNA was extracted using a small amount extraction kit (Magen, R416503). For details, see www.magentec.com.cn.

2) Agarose gel electrophoresis: Analysis of RNA integrity and DNA contamination of samples.

3) NanoPhotometer spectrophotometer: Determination of RNA purity (OD260/280 and OD260/230 ratio).

### (4) Construction, Sequencing and Data Analysis of Small RNA Libraries

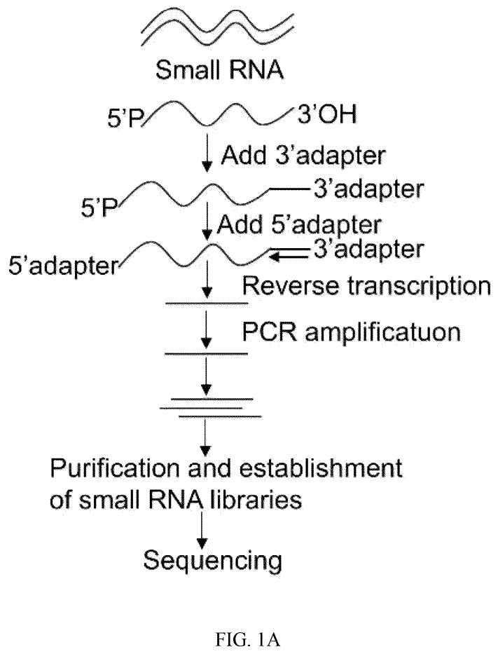

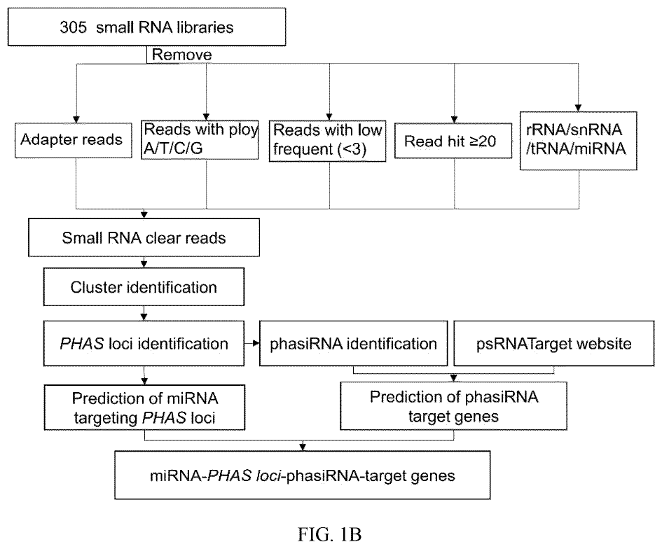

After 24 hours of Pb treatment, three plants from each inbred line were randomly selected, and their roots were extracted for RNA extraction and sequencing to construct a small RNA library. Beijing Novogene Technology Co., Ltd. (https://www.novogene.com/) was commissioned to complete the library construction and sequencing of the small RNA from the maize inbred line population. The sequencing was performed using the Illumina HiSeq2000 instrument. The specific sequencing and data analysis processes are illustrated in FIG. 1A. Once the samples were verified to meet the quality criteria (RIN >7), the unique structures present at the 3′ and 5′ ends of the small RNA, specifically, a hydroxyl group at the 3′ end and a complete phosphate group at the 5′ end were utilized to directly attach adapters to both ends of the small RNA. This was followed by reverse transcription into cDNA. Subsequently, PCR amplification and PAGE gel electrophoresis were employed to separate the target fragments. The cDNA library was recovered through gel cutting and then sequenced using a computer to obtain high-quality sequencing data for the subsequent identification of PHAS loci.

Phasing analysis of the secondary siRNAs was performed according to previously described methods (Axtell, 2010; Guo et al., 2015). The psRNATarget prediction website was employed to identify miRNAs that target the PHAS loci. The processes were illustrated in FIG. 1B.

Identification of PHAS loci and phasiRNA
1) Identification of phasiRNA Cluster

Initially, we processed the raw data from the small RNA database by removing adapters and filtering out low-frequency reads (those with fewer than three occurrences) and multi-site matches (those with more than 20 hits). We included reads from playA/T/G/C, as well as rRNA, snRNA, tRNA, miRNA, and other relevant categories to obtain valid data (clean reads). The current study specifically identifies PHAS loci that generate 21 nt sequences; therefore, only 21 nt clean reads were selected for subsequent analysis. These filtered 21 nt clean reads were aligned to the maize B73 V4 reference genome using Bowtie, and information regarding the starting position, abundance, strand orientation (both positive and negative), and chromosome location of the matched small RNAs was recorded. We excluded loci that mapped to repetitive regions to minimize background noise. Subsequently, small RNA clusters within the genome were identified, and the starting positions of these small RNAs were documented.

A. Each read length is 21 nt, and the spacing between reads groups should be a multiple of 21. Reads groups are ordered according to their positions on the genome from smallest to largest, P1, P2, P3 . . . Pn.

B. Pn-Pn 1≤84 nt, and there are more than 4 consecutive reads, then the largest continuous segment that meets these conditions will be regarded as the phasiRNA cluster.

### 2) Screening and Calculation Method of PHAS Gene

The unique 21-nt sRNA sequences were mapped to the B73 RefGen_v4 genome, allowing for no mismatches (Xia et al., 2015; Sablok et al., 2018). Phasing analysis of the secondary siRNAs was conducted following previously established methods (Axtell, 2010; Guo et al., 2015). Each identified phasiRNA cluster was categorized into 21 bins. The abundance of sRNAs in each bin (Bin_Abu) and in each cluster (Cluster_Abu), as well as the number of specific sRNAs in each bin (Bin_Num) and the bin length (bin_length), were obtained. The calculation equations are as follows:

$$Phase\_ratio = unique number of sRNA \times 21 / bin\_length ; \qquad ( i )$$

$$phase score = Abu\_ratio \times Bin\_Num \times ln ( Bin\_Abu ) ; \qquad ( ii )$$

$$Abu\_ratio = Bin\_Abu / Cluster\_Abu . \qquad ( iii )$$

Among them, phase_ratio primarily represents the proportion of specific small RNAs within each bin, reflecting the degree to which these small RNAs conform to the phase arrangement. A larger phase_ratio indicates a more pronounced characteristic of the phase arrangement in the small RNA head-to-tail connections. Abu_ratio denotes the proportion of small RNA abundance in a bin relative to the overall abundance within the identified Cluster. A higher Abu_ratio suggests that the abundance of small RNA in the bin is greater compared to other bins within the Cluster, indicating that the corresponding site may represent a genuine PHAS locus. Phase_score serves as a comprehensive metric that integrates both small RNA phase arrangement and abundance. Consequently, the number of small RNAs across the 21 bins of each cluster was quantified, and the phase_ratio for each bin was calculated, and sorted in descending order. Additionally, the abundance of differential small RNAs across the 21 bins in each cluster was statistically analyzed, and the Abu_ratio for each bin was computed and sorted in descending order as well. Finally, the comprehensive scores, which combine the phase_ratio and Abu_ratio rankings, were organized from smallest to largest. Sites with lower comprehensive scores are more likely to represent PHAS loci that generate phasiRNAs.

### (5) Pb Content Determination

1) The dried samples were crushed, and 0.2 g of the sample powder was weighed into a microwave digestion tube. Each sample was prepared with three biological replicates, followed by the addition of 10 ml of high-purity nitric acid.

2) The digestion tube was placed in microwave digester, and the plant digestion program was selected, which involved heating to 80° C. for 5 minutes, maintaining that temperature for an additional 5 minutes, and then increasing the temperature to 180° C. for 20 minutes, at which point digestion was considered complete.

3) After opening the cover, the digestion tube was transferred to an intelligent temperature-controlled electric heater (PreeKem, G400), and heated at 180° C. until the remaining liquid volume was approximately 0.5 mL.

4) The remaining liquid was filtered using filter paper and washed three times with ultra-pure water. The collected liquid was then adjusted to final volume of 10 mL.

5) Prior to measurement, the ICP MS instrument was calibrated and tuned with tuning fluid to ensure optimal performance and enhance the accuracy of the measurement data.

6) Following the instrument tuning, a standard curve was prepared with seven concentrations of Pb solutions, specifically 5, 10, 20, 40, 60, 80 and 100 ng/mL. The correlation coefficient of the standard curve was maintained at no less than 0.999.

7) The Pb content of the sample was calculated based on the concentration and mass of the sample.

### 1.2 Results

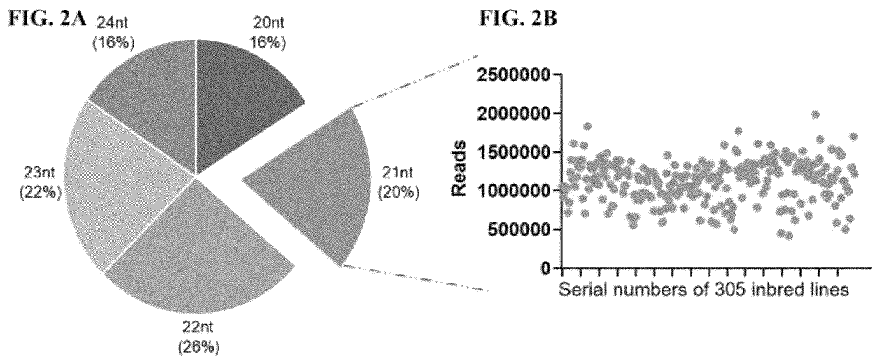

To profile phasiRNAs involved in the maize response to Pb stress, we conducted sRNA sequencing on the seedling roots of 305 maize lines following 24 hours of Pb treatment. In this study, the 305 sRNA libraries were combined to identify PHAS loci and phasiRNAs. After excluding reads that matched rRNA, tRNA, snRNA, snoRNA, and miRNA sequences, we retained 1.77×10⁹ clean reads with size ranging from 20 to 24 nt. Specifically, 16%, 20%, 26%, 22%, and 16% of these reads were 20, 21, 22, 23, and 24 nt sRNAs, respectively (FIGS. 2A and B). By processing the sequenced reads and phasing analysis with a sequenced reads and performing the threshold of phase score ≥25 and phase_ratio ≥0.5 (Dotto et al., 2014), 1,161,927 phasiRNAs (21 nt) were generated from 137 PHAS loci, among which 71 PHAS loci were located within gene models. However, some PHAS loci are typically embedded within large windows, leading to an uncharacteristically high number of unique sRNA reads and an inflation of the phase score (Dotto et al., 2014). Consequently, these PHAS loci are unlikely to be associated with distinct PHAS loci. To reduce the false positive rate, we established a stricter threshold of Abu_ratio (Abundance ratio, bin abundant/cluster abundant) ≥0.3 for identifying the PHAS loci. This resulted in the identification of 22 PHAS loci situated within gene models and the derivation of 423 unique phasiRNAs.

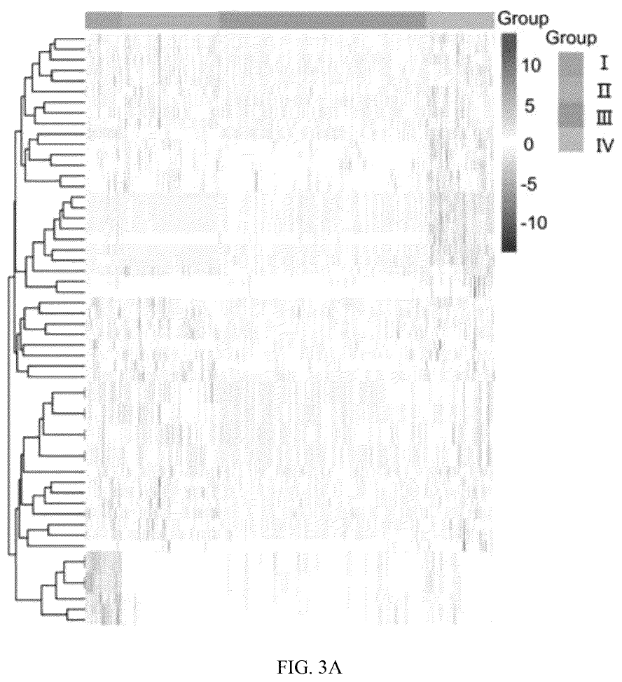

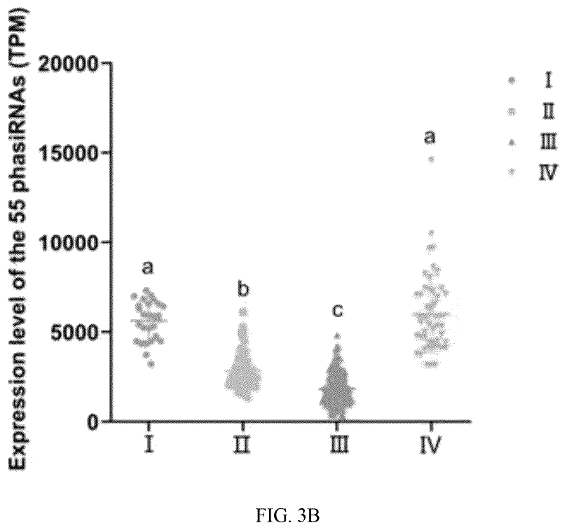

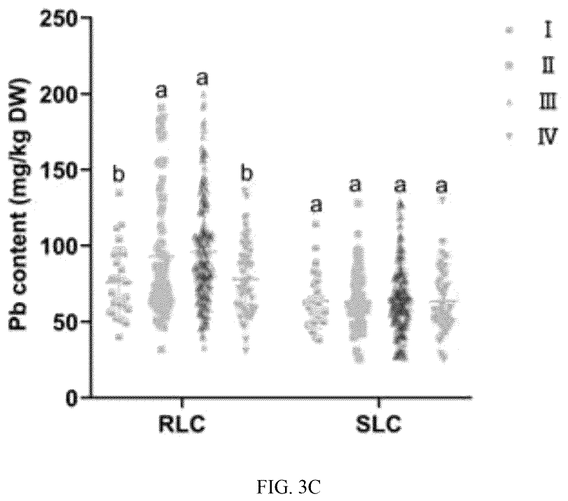

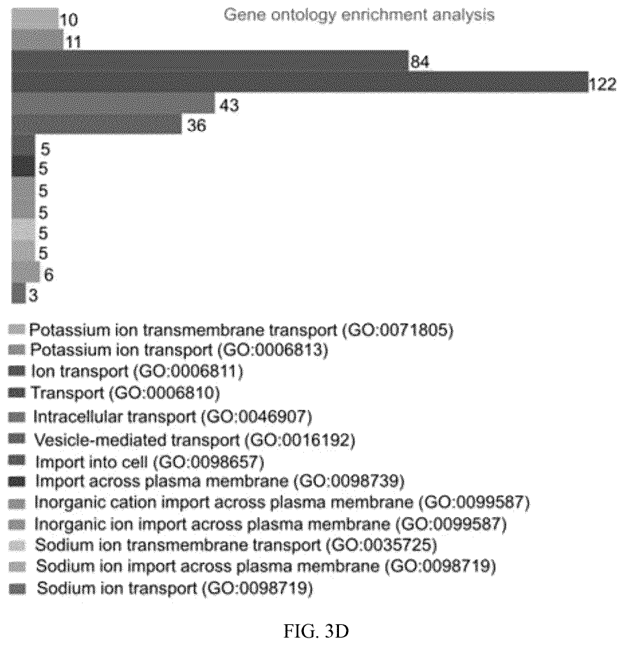

By referring to the previous study (Luo et al., 2022), phasiRNAs with an average RPM (reads per million mapped reads) across 423 lines ≥3 were defined as high-abundance phasiRNAs and retained in the present study. Finally, 55 phasiRNAs derived from 13 PHAS loci remained (Table 2). Based on the expression levels of the 55 phasiRNAs, the 305 lines were divided into four groups using the K-mean clustering algorithm (I, n=27; II, n=73; III, n=154; IV, n=51) (FIG. 3A). The expression levels of phasiRNAs in Group I and IV were significantly (P<0.01) higher than those in Group II and III, whereas the Pb content in the roots of Group I and IV was significantly (P<0.01) lower than that in Group II and III (FIGS. 3B and 3C). These suggest that the expression levels of phasiRNAs affect the Pb content in maize seedling roots.

TABLE 2
PHAS loci deriving high-abundance phasiRNAs

| Gene model | BinPosition | Bin_Length | Bin_Num | Bin_Abu | Cluster_Num | Cluster_Abun |
|---|---|---|---|---|---|---|
| Zm00001d028925 | 1-51729712- | 648 | 23 | 80670 | 374 | 91174 |
| | 51730359 | | | | | |
| Zm00001d004867 | 2-145453799- | 879 | 25 | 19691 | 413 | 36081 |
| | 145454677 | | | | | |
| Zm00001d041491 | 3-123759979- | 50 | 12 | 1026 | 82 | 1317 |
| | 123760479 | | | | | |
| Zm00001d053394 | 4-229142093- | 1509 | 46 | 24036 | 874 | 41875 |
| | 229143601 | | | | | |
| Zm00001d051054 | 4-139211490- | 291 | 13 | 3558 | 81 | 4222 |
| | 139211780 | | | | | |
| Zm00001d014377 | 5-43571895- | 585 | 34 | 984 | 193 | 1511 |
| | 43572479 | | | | | |
| Zm00001d035601 | 6-36081583- | 690 | 16 | 1976 | 182 | 6149 |
| | 36082272 | | | | | |
| Zm00001d018725 | 7-3139002- | 648 | 20 | 18587 | 401 | 32683 |
| | 3139649 | | | | | |
| Zm00001d008099 | 8-134118014- | 480 | 25 | 13926 | 314 | 18618 |
| | 134118493 | | | | | |
| Zm00001d010936 | 8-134118014- | 669 | 31 | 13946 | 339 | 18689 |
| | 134118493 | | | | | |
| Zm00001d012477 | 8-175269942- | 501 | 14 | 671 | 103 | 979 |
| | 175270442 | | | | | |
| Zm00001d027187 | 9-136742674- | 900 | 42 | 135419 | 579 | 160207 |
| | 136743573 | | | | | |
| Zm00001d023134 | 10-85278699- | 396 | 22 | 34855 | 252 | 42278 |
| | 85279094 | | | | | |

Among the 55 phasiRNAs, 41 derived from nine PHAS genes were significantly negatively correlated with Pb content (P<0.05, correlation coefficient: −0.141 to −0.328). The predicted targets of the associated phasiRNAs were significantly enriched in ion transport and import, including potassium ion transmembrane transport, potassium ion transport, ion transport, and inorganic ion import across plasma membrane (FIG. 3D). This implies that phasiRNAs play a significant role in ion transmembrane transport and import via post-transcriptional regulation.

2. The Results of eGWAS Analysis Showed that PHAS_1 was Important in Lead Tolerance of Maize

### 2.1 Materials

The MaizeSNP50 BeadChip, which encompasses 56,110 SNPs, was previously used to genotype 305 inbred maize lines. In the present study, a total of 43,668 high-quality SNPs remained for the eGWAS after excluding SNPs with missing data (>20%), minor allele frequency (<0.05) or heterozygosity (>20%). The expression levels of the nine PHAS genes, which negatively correlate with Pb content were extracted from the processed sRNA sequencing libraries of the 305 lines. The MLM (Q+K) was employed for the eGWAS to identify the associations between each SNP and the expression levels of these PHAS genes. The significance threshold was established at P=0.05/SNP number=0.05/43,668=1.14×10⁻⁶.

### 2.2 Results and Analysis

2.2.1 eGWAS of PHAS Genes

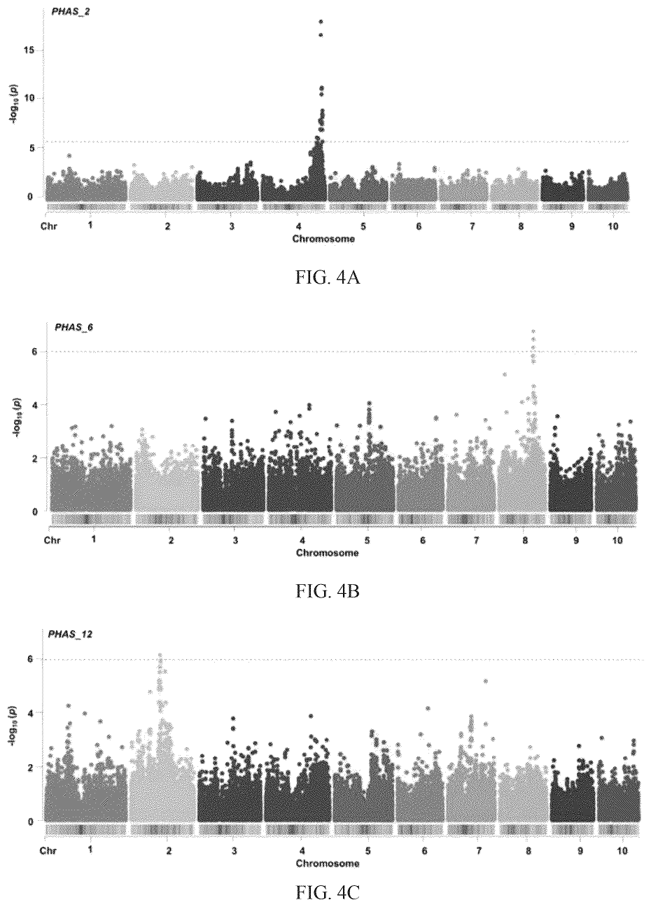

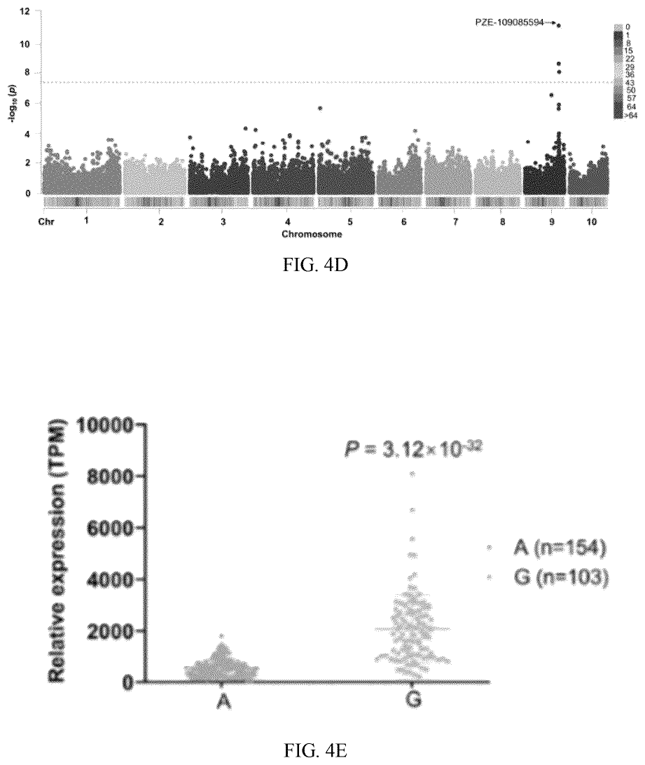

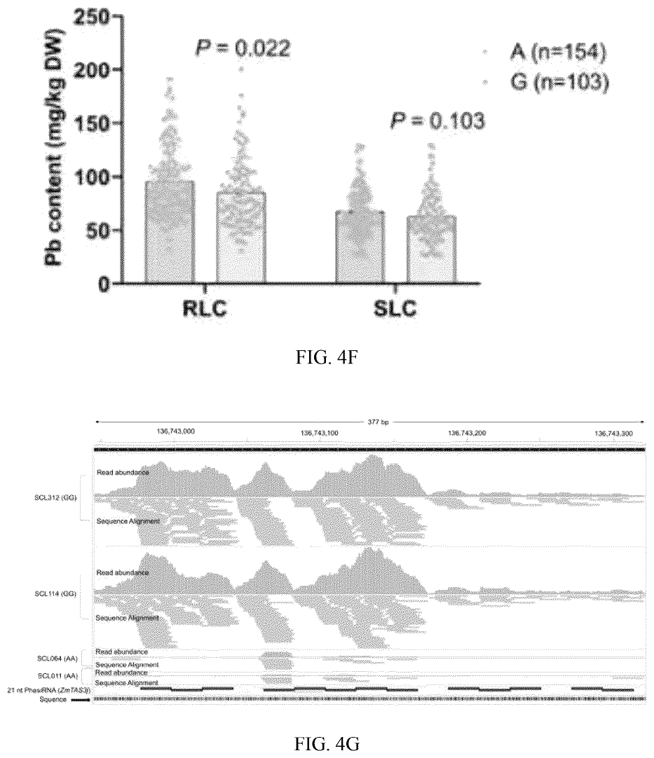

In this study, a total of 9 PHAS genes were identified to participate in the Pb stress response of maize. To identify the genetic loci controlling the expression of these nine PHAS genes under Pb treatment, we performed an eGWAS, integrating 43,668 high-quality single nucleotide polymorphisms (SNPs) with the expression levels of the PHAS genes in this maize panel. Ultimately, we detected 31 SNPs that met the threshold of P=0.05/SNP number (1.14×10⁻⁶) using the Mixed Linear Model (MLM). These SNPs were associated with the expression levels of PHAS_1 (3 SNPs), PHAS_2 (22 SNPs), PHAS_6 (3 SNPs), and PHAS_12 (1 SNP), respectively (FIGS. 4A, B, C, and D). However, no significant SNPs were identified for the remaining five PHAS genes. According to the previous study (Fu et al., 2013), at least three significant SNPs located within an interval of less than 5 kb and associated with a trait were classified as an eQTL. In our analysis, we identified only one candidate eQTL (eQTL1), which included three significant SNPs (PZE-109085571, PZE-109085594, and SYN21521) associated with the expression abundance of PHAS_1. Notably, eQTL1 was located precisely within PHAS_1, with the three SNPs representing exon and downstream variations, respectively. Among these, PZE-109085594 exhibited the lowest P-value (7.36×10⁻¹²) and was thus designated as the lead SNP of eQTL1. Based on the lead SNP, the maize lines were categorized into two primary groups: one group (154 lines) containing the A allele and the other group (103 lines) containing the G allele. A t-test revealed that the A-containing group had a significantly lower expression (mean=551.64 transcripts per million, TPM) compared to the G-containing group (mean=2092.13 TPM) of the G-containing group with P=3.12×10⁻³² (FIG. 4E). The Pb content of the A-containing group was significantly (P=0.022) higher than that of the G-containing group (FIG. 4F). These results suggested that eQTL1 regulated the expression of PHAS_1 and thus affected Pb content performance in maize seedings.

### 2.2.2 PHAS_1 was a New Member of the TAS3 Gene Family (ZmTAS3j)

Among the phasiRNAs derived from PHAS_1, 13 phasiRNAs were retained, demonstrating a significant negative correlation with the Pb content in maize roots. Specifically, the correlation coefficients for phasiRNA_136742998, phasiRNA_136743059, and phasiRNA_136743017 with the Pb content of maize seedling roots were 0.328 (P=2.88×10⁻⁶), 0.318 (P=6.02×10⁻⁶), and 0.304 (P=1.55×10⁻⁵), respectively. Concurrently, the target genes of tasiRNA derived from PHAS_1 were predicted using the http://plantgrn.noble.org/psRNATarget/website. Notably, phasiRNA_136743059 and phasiRNA_136743080, located in the 5th and 6th phases, target multiple ZmARF genes, including Zm00001d039006, Zm00001d043922, Zm00001d012731, Zm00001d038698, Zm00001d043431, and Zm00001d042267. Among these, Zm00001d039006, Zm00001d043922, Zm00001d012731, and Zm00001d038698 belong to the ZmARF3 gene family. Therefore, PHAS_1 conform to the characteristics of the TAS3 gene family and has been designated as ZmTAS3j.

3.ZmTAS3j was Targeted by miR390

### 3.1 Materials and Methods

### 3.1.1 Materials

The seeds of N. benthamiana were preserved by our research group and cultured in the plant culture room at Sichuan Agricultural University. The expression vectors pCAMBIA2300 35S eGFP and pRI101 AN were also preserved by the research team. The Agrobacterium used for transformation, GV3101, was purchased from Shanghai Weidi Biotechnology Co., Ltd.

### 3.1.2 Methods

(1) Construction of Expression Vector of ZmTAS3 and eGFP.

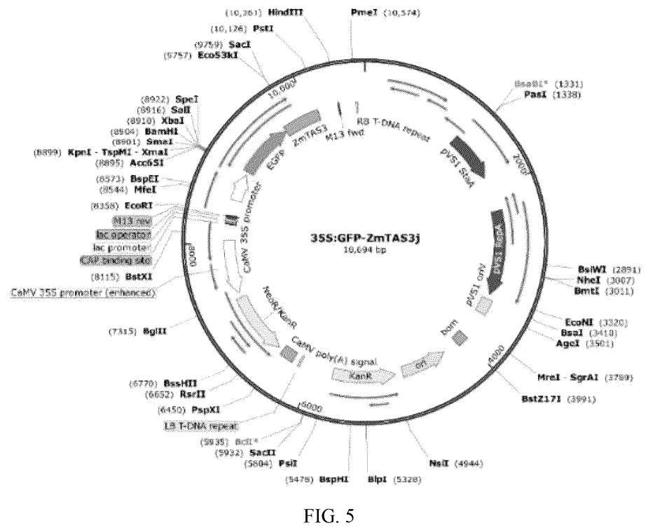

The pCAMBIA2300-35S-eGFP vector was used as the base vector, and the fragment of ZmTAS3j was constructed by homologous recombination method using BsrGI(TGTACA) single enzyme digestion, named 35 s: eGFP ZmTAS3j. The vector map as shown in the FIG. 5. The sequence of GFP-ZmTAS3j-F: tcggca tggacgagctgtacaGCGGTTTCGTTCTCCTTCCT; The sequence of GFP-ZmTAS3j-R: atgcctgcagttacttgtacaCTCAA CACAGCTCAGAAGGGAT.

### (2) Construction of OE-miR390 and STTM390 Vector.

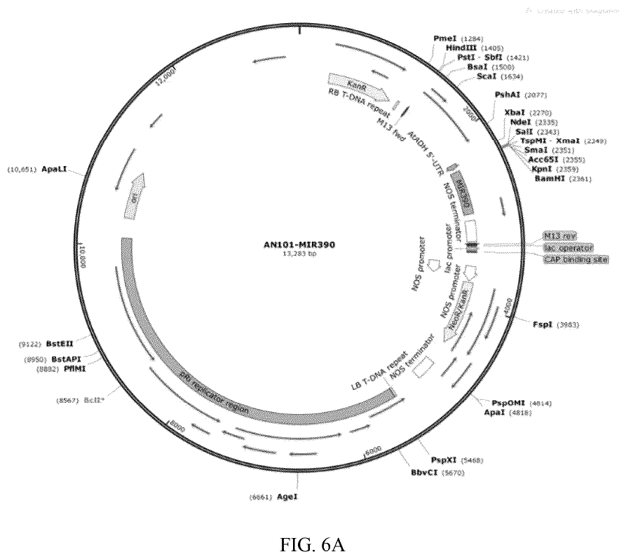

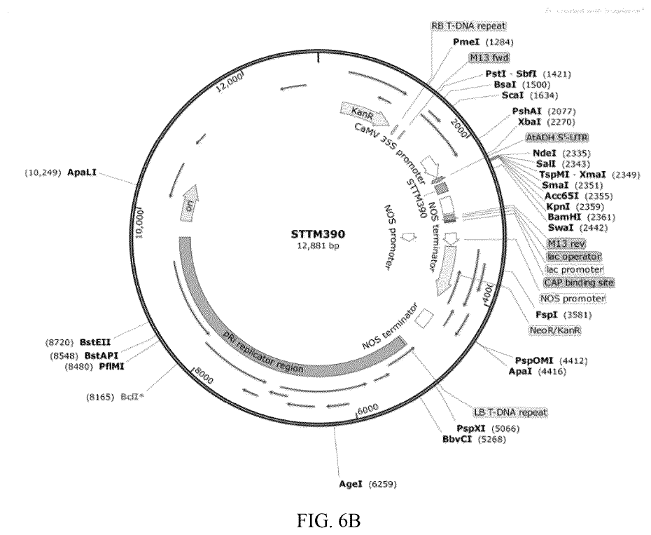

The pRI101-AN vector was used as the base vector. The fragment of MIR390 was constructed by homologous recombination method using BsrGI(TGTACA) single enzyme digestion, named as 35S:MIR390. The vector map as shown in the FIG. 6A. The sequence of AN101-miR390-F gggggtaccggatccgaattcCATTCCCATCCGTTCCTGCT; The sequence of AN101-miR390-R: agagttgttgattcagaattcGCGTGTACCTGACGAGAGAC

A 146 bp short tandem target mimic (STTM) fragment containing two miR390 target sites was synthesized by Qingke Biotech. This fragment incorporates three base mismatches at the targeted cleavage site of miR390, which generate structural bubbles that hinder the cleavage of miR390. Consequently, this mechanism promotes the adsorption of miR390 and inhibits the targeted cleavage of the gene ZmTAS3j by miR390. The STTM390 (146 bp) sequence was integrated into the pRI101 AN basic expression vector using EcoRI (GAATTC) as the sole restriction enzyme, via homologous recombination. This construct was designated as 35S: STTM390. The vector map for the STTM is illustrated in FIG. 6B. STTM390 fragment sequence.

catttggagaggacagcccaagcttGGCGCTATctaTCCTGAGCTTgttg
ttgttgttatggtctaatttaaatatggtctaaagaagaagaaTGGCGCT
ATctaTCCTGAGCTTgaattcggtacgctgaaatcaccag.

(3) Transient Expression System of Nicotiana benthamiana Confirmed that miR390 Targeted Regulation of ZmTAS3j Expression

The 35S:eGFP-ZmTAS3j, AN101-MIR390, and AN101-STTM390 vectors were transformed into Agrobacterium tumefaciens GV3101, followed by bacterial liquid PCR to identify positive clones. The N. benthamiana transient expression system was utilized to verify the targeted cleavage of ZmTAS3j by miR390. If ZmTAS3j is indeed targeted and cleaved by miR390, the expression of the eGFP protein will gradually diminish as the concentration of miR390 increases. When the STTM390 bacterial solution is introduced, miR390 is sequestered by the STTM390 sequence, leading to a reduction in the targeted cleavage of the ZmTAS3j gene by miR390. This reduction subsequently alleviates the inhibition of eGFP expression and enhances fluorescence intensity. The procedure for the Nicotiana benthamiana transient expression was as follow:

1) Select the transformed positive clones and add 1 ml of YEP medium containing the corresponding antibiotics (Kan and Rif, at a concentration ratio of 1:1000). Culture the cells at 28° C. with a shaking speed of 250 rpm for 16 hours.

2) Subsequently, transfer the Agrobacterium culture to an Erlenmeyer flask containing 50 ml of resistant medium (Kan and Rif, at a concentration ratio of 1:1000) and expand the culture overnight (approximately 16 to 18 hours). Once the optical density (OD) of the bacterial solution reaches approximately 1.0, centrifuge the culture at 4000 rpm for 10 minutes at room temperature and collect the bacterial pellet.

3) Resuspend the pellet in a solution of 10 mM MgCl2, supplemented with MES and AS at a ratio of 1:1000, and adjust the concentration of the Agrobacterium containing the 35S:eGFP-ZmTAS3j vector to an OD600 of 0.6. The concentration of the 35S:MIR390 Agrobacterium will be established in three gradients (OD600 values of 0.6, 1.0, and 1.4, respectively), while the Agrobacterium OD600 for the STTM390 expression vector will be adjusted to 1.0.

4) Utilize N. benthamiana plants that are approximately one month old and not yet flowering. Mix the different concentrations of miR390 bacterial solutions (OD600 0.6, 1.0, and 1.4) with the target gene bacterial solution (OD600 0.6) in equal proportions and inject this mixture into the leaves of N. benthamiana.

5) Additionally, mix the miR390 solution at a concentration of 1.0 with the target gene solution at a concentration of 0.6, along with the Agrobacterium concentration of 1.0 from the STTM390 expression vector, in equal proportions for injection into N. benthamiana.

6) After injection, place the treated N. benthamiana. in a culture room, and after 48 hours, observe eGFP fluorescence using a confocal microscope (LSM 800, ZEISS, Germany).

(4) 5′RACE verified the targeted site of miR390 mediated ZmTAS3j.

In this study, the RLM RACE Kit (Invitrogen) was utilized to verify the specific cleavage site of the ZmTAS3j gene by miR390. The target fragment of the 5′ RACE is a degradation fragment resulting from the cleavage of the target gene by miRNA. Consequently, the 5′ end of these degradation fragments lacks a cap structure and possesses an exposed monophosphate group, thereby eliminating the need for removal prior to the addition of an adapter at the 5′ end. For capping and dephosphorylation, the remaining steps were carried out according to the kit instructions. Nested PCR primers were designed as follows: RACE-out: TCAGCCACCATCTTTATCCTT CCTCA and RACE-inner: AGATCAGGTCTTCTTGACCTTGCA, based on the target gene sequence. PCR amplification was performed, followed by gel extraction of the expected single fragment using the gel recovery kit (Hipure Gel Pure DNA Mini kit, Magen) for analysis of the PCR product. The gel recovery product was subsequently sent to Blant for sequencing.

(5) ZmTAS3j-Derived tasiARFs Cleave ZmARF3

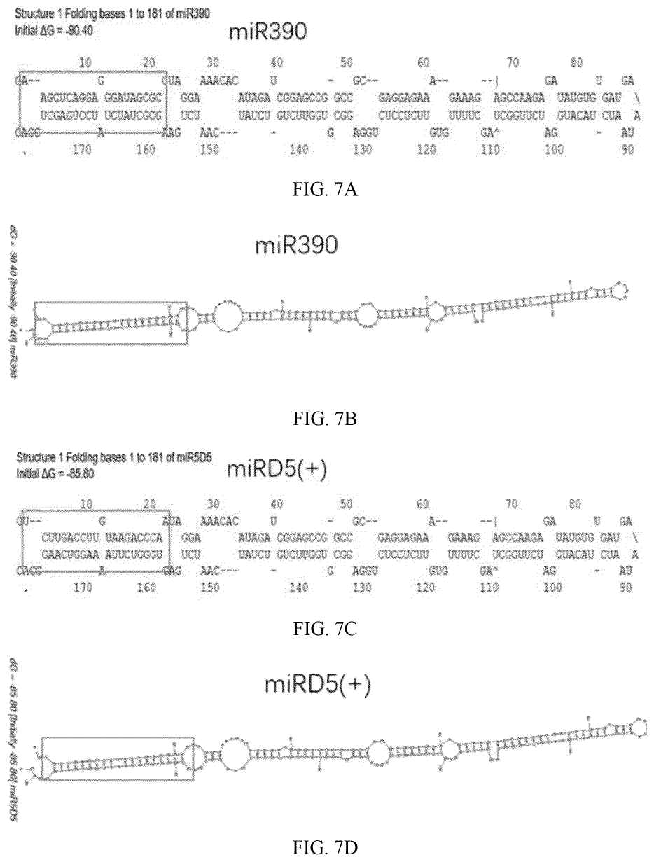

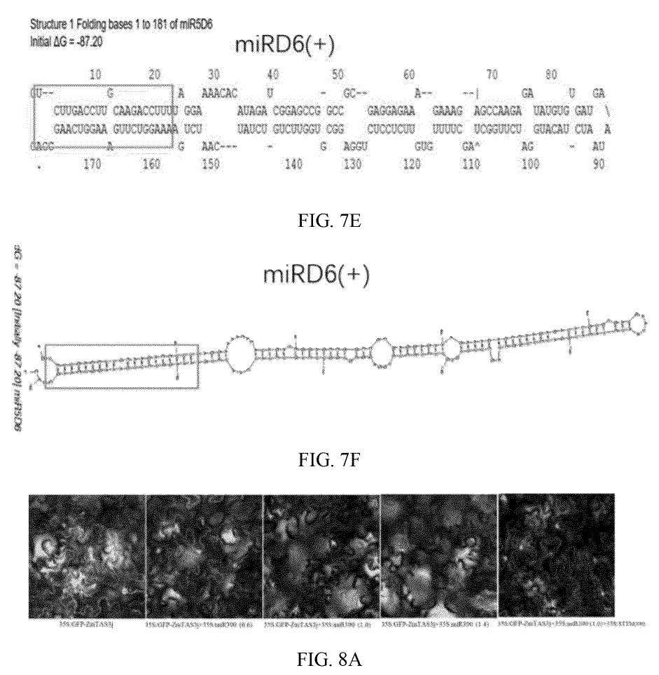

Referring to the method of transforming the miRNA precursor structure as described by Montgomery et al., based on the MIR390 sequence, the mature sequence of miR390 was substituted with tasiD5(+) and tasiD6(+). Additionally, the sequence of miR390 was replaced with tasiD5(+) and tasiD6(+). The complementary sequences of miR D5(+) and miR D6(+) were constructed through sequence modification. For further details, see FIG. 7. A sequence comparison of the ZmARF3 gene family revealed that the ZmARF3 family gene contains two tasiARF target recognition sites, which are relatively conserved in their sequences. The target fragment was obtained by PCR amplification using maize B73 cDNA as a template, and a gel recovery kit was employed to recover the target fragment. For specific operational steps, refer to the kit instructions (Hipure Gel Pure DNA Mini kit, Magen). Subsequently, the pCAMBIA2300 35S eGFP expression vector was utilized as the basic vector. Using the enzyme cutting site BsrGI (TGTACA) for single enzyme digestion, the conserved ARF3 gene sequence was constructed into the pCAMBIA2300 35S eGFP expression vector via homologous recombination, resulting in the vector named pCAMBIA2300.35S eGFP ZmARF3. The pCAMBIA2300 35S eGFP ZmARF3 Agrobacterium strain was co-injected into tobacco leaves alongside miR D5(+) or miR D6(+), and the pCAMBIA2300 35S eGFP ZmARF3 Agrobacterium strain was also injected into tobacco leaves independently. The injected tobacco was subsequently placed in the plant culture room at Sichuan Agricultural University, where eGFP fluorescence was observed using a fluorescence confocal microscope (LSM 800, ZEISS, Germany) after a 48-hour incubation period.

(6) RNA Extraction, Reverse Transcription and Quantification of ARF3 mRNA.

N. benthamian leaves infected with Agrobacterium for 48 hours were collected and ground in liquid nitrogen. RNA was extracted using a plant total RNA mini-extraction kit (Magen, R4165 03). For detailed operating procedures, refer to the instruction manual available at www.magentec.com.cn. The reverse transcription kit (First Strand cDNA Synthesis Kit, Novoprotein) was employed to synthesize the first strand of cDNA. Forward and reverse primers were designed, and the quantitative analysis of ARF3 was conducted using the quantitative kit (SYBR qPCR SuperMix Plus, Novoprotein). The specific experimental system is detailed in the kit manual. The tobacco 18S gene served as the internal reference gene, with the following quantitative primers: AtARF3-F: AGGTTCTGCATCACCCTCAC, AtARF3-R: GGTTCACGCCAGTAGCATCT, AtActin-F: TGGAATCCACGAGACAACCTA, and AtActin-R: TTCTGTGAACGATTCCTGGAC. All experimental analyses were performed with three biological replicates, and Excel was utilized to analyze the relative expression levels of the genes.

### 3.2 Results and Analysis

3.2.1 ZmTAS3j was Targeted by miR390

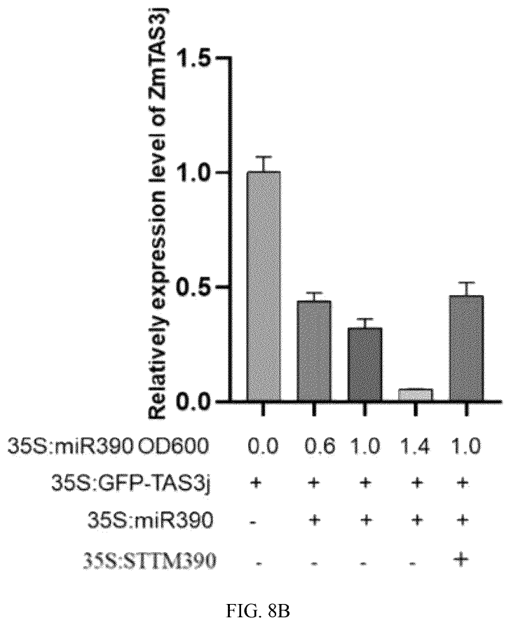

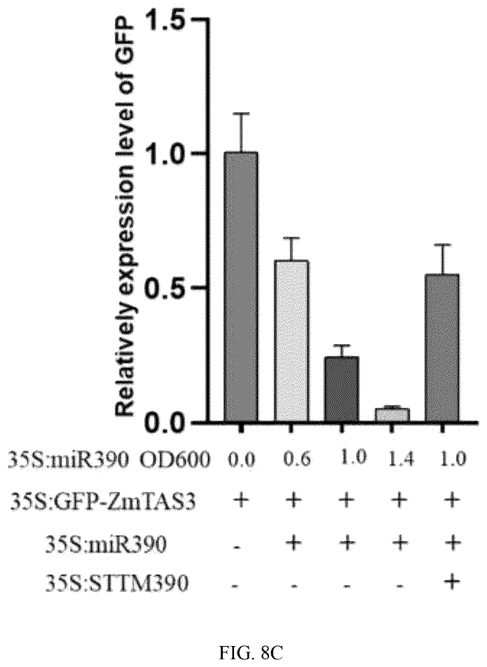

To confirm whether ZmTAS3j was cleaved by zma-miR390, we constructed the recombinant vector 35S:eGFP-ZmTAS3j and co-transformed it with the expression vector of zma-miR390 (35S:miR390) into N. benthamiana leaves. The eGFP fluorescence intensity in the transformed N. benthamiana leaves decreased with increasing concentrations of zma-miR390 (OD600ₙₘ: 0, 0.6, 1.0, and 1.4) (FIG. 8A). Furthermore, we designed a zma-miR390 target mimicry construct (35S:MIM390) that contained two uncleavable zma-miR390-binding sites linked by a 48-nt spacer. The fluorescence intensity increased in the N. benthamiana leaves co-transformed with 35S:eGFP-ZmTAS3j, 35S:miR390, and 35S:MIM390, compared to those co-transformed with only the 35S:eGFP-ZmTAS3j and 35S:miR390 vectors (FIG. 8A). Additionally, the mRNA abundances of ZmTAS3j and GFP in the N. benthamiana leaves were consistent with the fluorescence observations (FIGS. 8B and C). These results confirmed that zma-miR390 induced target cleavage of ZmTAS3j mRNA.

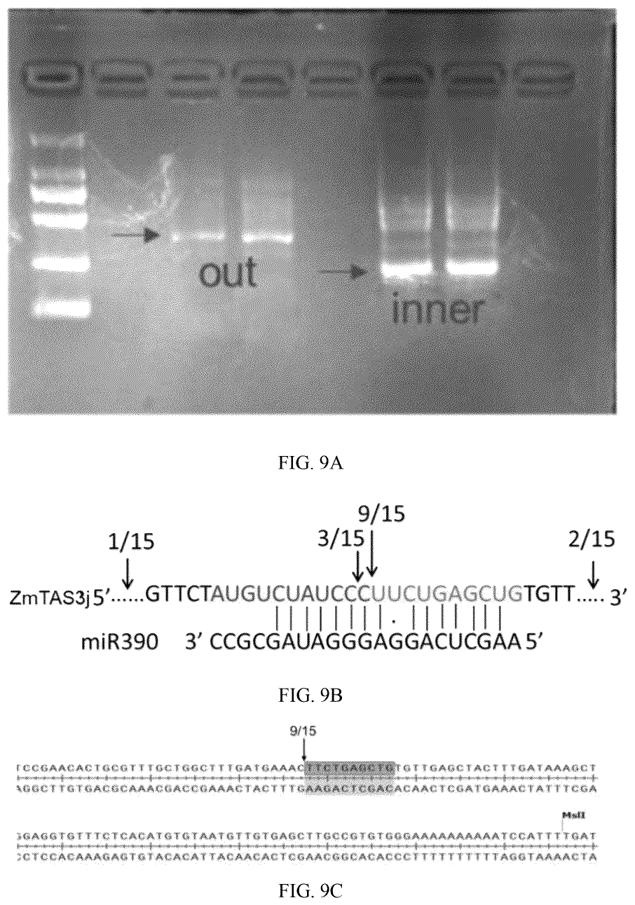

Furthermore, the 5′ rapid amplification of cDNA ends (RACE) was employed to confirm the specific location where miR390 mediates the targeted cleavage of ZmTAS3j. Initially, RNA was extracted from tobacco leaves co-expressing 35S:GFP-ZmTAS3j and miR390 Agrobacterium. Following the protocol of the 5′ RACE kit, T4 ligase was utilized to attach the 5′ adapter sequence, after which random primers facilitated the reverse transcription of the sequence into cDNA. Specific primers were designed based on the adapter sequence and the ZmTAS3j sequence, and nested PCR was conducted to amplify the specific band (FIG. 9A). Subsequently, the product was ligated and transformed into E. coli, from which 15 single clones were randomly selected for sequencing. The sequencing results indicated that the targeted cleavage position of ZmTAS3j by miR390 predominantly occurred between the 10th and 11th bases (9 of 15 5′-RACE products) at the 3′ end of the miR390 target site, a characteristic position for miRNA-mediated cleavage (FIGS. 9B and C).

3.2.2 miR390 Targets ZmTAS3j and Generates tasiRNA in the 5′ direction

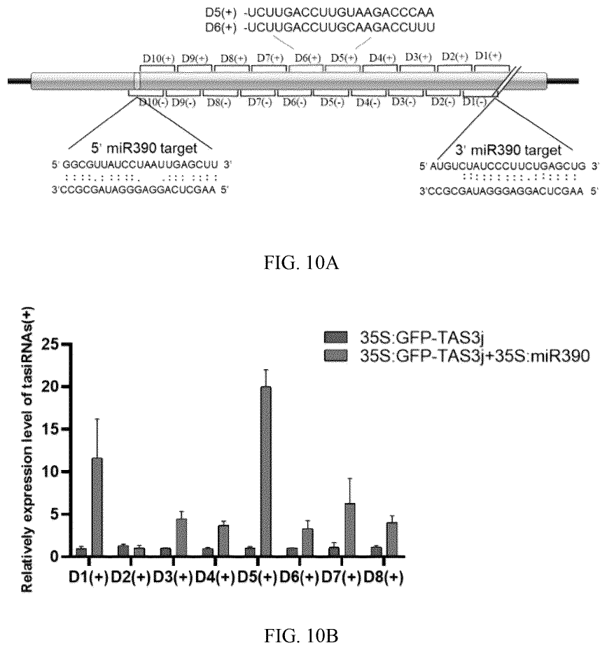

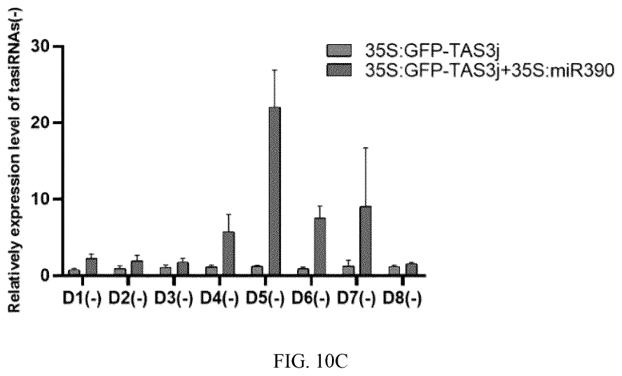

Targeting TAS3 family genes by miR390 exemplifies a typical “two-hit” mechanism, wherein the sequence between the two target sites serves as the precursor for tasiRNA production. The aforementioned 5′ RACE experiment confirmed that the cleavage site occurs between the 10th and 11th bases at the 3′ end of the miR390 target site. DCL4 initiates processing from this cleavage site along the 5′ end of the sequence, resulting in the generation of a series of phase-aligned tasiRNAs (FIG. 10A). Bioinformatics analysis of the small RNA sequencing database revealed that perfectly phase-aligned tasiRNAs can be produced at this cleavage site, with a high abundance of tasiRNA detected. The expression levels of tasiRNAs were further validated through small RNA quantitative analysis. When 35S:eGFP-ZmTAS3j and miR390 were co-expressed in tobacco leaves, the expression levels of multiple tasiRNAs were significantly elevated compared to those in tobacco leaves expressing only 35S:eGFP ZmTAS3j. Notably, tasiARF (D5(+)) exhibited the highest expression abundance (FIGS. 10B and C).

3.2.3 ZmTAS3j-Derived tasiARFs Cleave ZmARF3

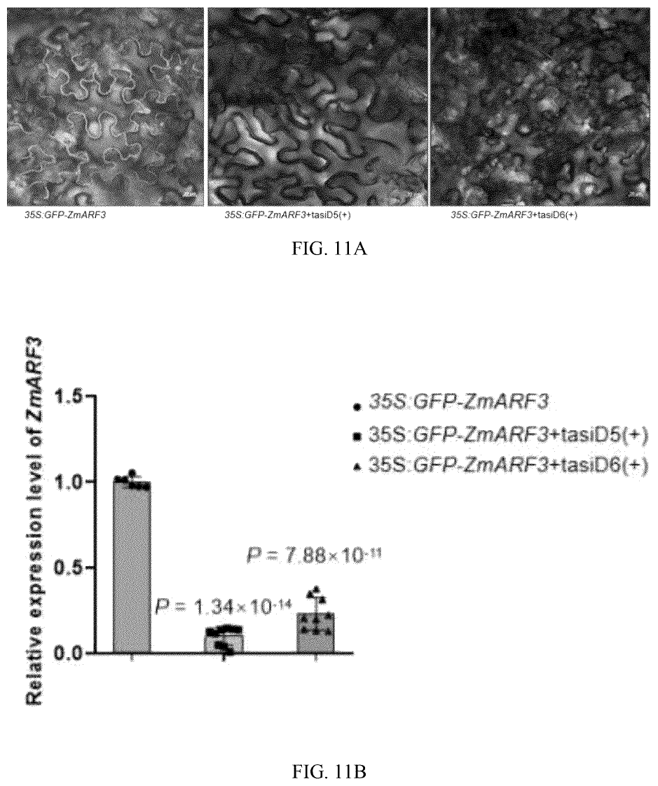

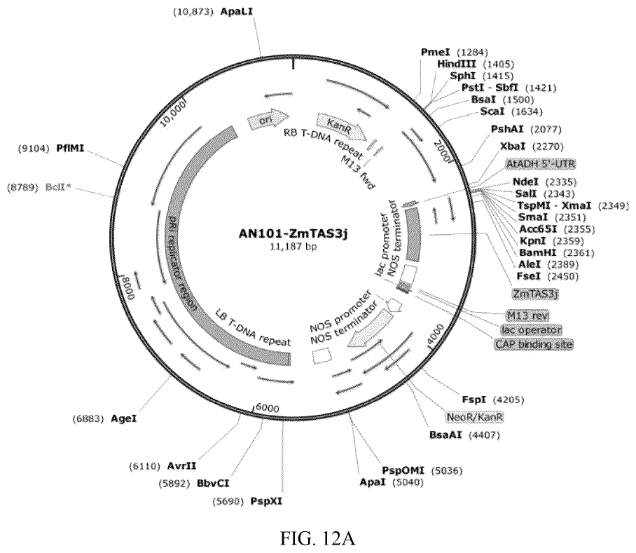

In the present study, ZmARF3 was expectedly targeted by tasiRNAs derived from the D5(+) and D6(+) positions of ZmTAS3j. We constructed the recombinant vector 35S:eGFP-ARF3, which included two target sites for tasiARFs, D5(+) and D6(+), within ZmARF3. Subsequently, we co-transfected 35S:GFP-ARF3 with either 35S:tasiD5(+) or 35S:tasiD6(+) into N. benthamiana leaves, resulting in reduced green fluorescence in the transformed leaves compared to the control (FIG. 11A). Furthermore, RT-qPCR analysis of the ZmARF3 transcript in the transformed N. benthamiana leaves corroborated the fluorescence observations (FIG. 11B). These results indicate that ZmARF3 is cleaved by ZmTAS3j-derived tasiARFs at the post-transcriptional level, ultimately leading to the degradation of ZmARF3 mRNA.

### 4. Overexpression of ZmTAS3j Enhanced Arabidopsis Tolerance to Pb Stress

### 4.1 Materials and Methods

### 4.1.1 Materials

The Colombian wild type (Col 0) Arabidopsis seeds used in this study were preserved in our laboratory, Escherichia coli Mach1-T1 was purchased from Beijing Baiao Leibo Technology Co., Ltd., Agrobacterium GV3101 was purchased from Shanghai Weidi Biotechnology Co., Ltd., and the pRI101 AN expression vector was preserved in our laboratory for a long time.

### 4.1.2 Methods

### (1) Construct Vector

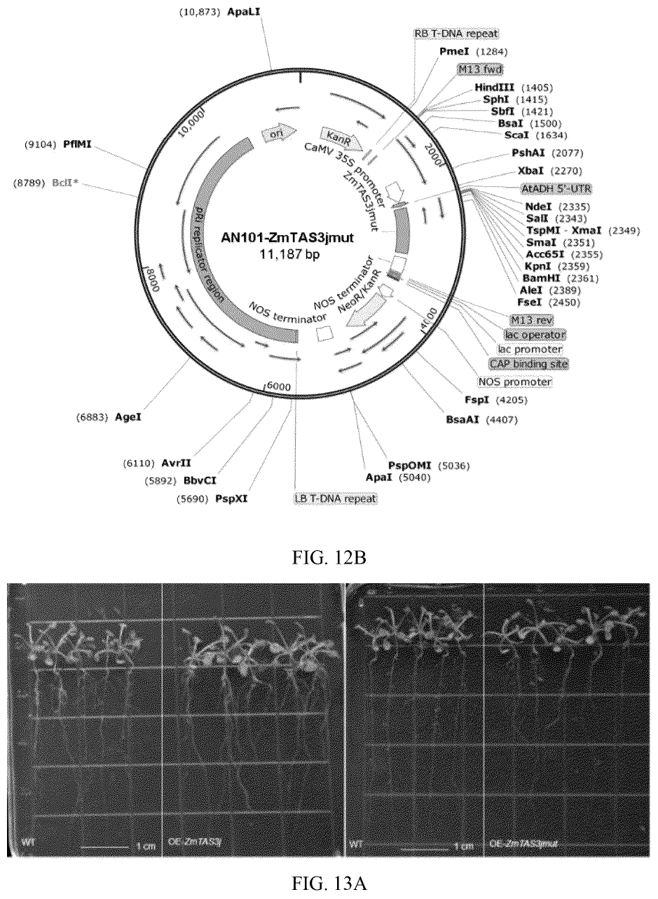

The total RNA from B73 inbred lines was extracted and reverse-transcribed into cDNA. The sequences of ZmTAS3j were amplified and cloned into the pRI101-AN expression vector, utilizing EcoRI as the restriction site. This vector was designated AN101-ZmTAS3j (FIG. 12A). The primers sequences as follows: AN101-ZmTAS3j-F: gggggtacc ggatccgaattcGCGGTTTCGTTCTCCTTCCT and AN101-ZmTAS3j-R: agagttgttgattcagaattcGCAGCT TATTCCATCGAGGC. The 5′ target of miR390 within the ZmTAS3j sequence was mutated through mismatch PCR amplification, effectively disrupting the 5′ target of miR390. The mutant sequence of ZmTAS3j was then cloned into the pRI101-AN expression vector, again using EcoRI as the restriction site. This vector was named AN101-ZmTAS3jmut (FIG. 12B). The primer sequences as follows:

ZmTAS3jmut-R,
aatttcctattgcggGTGCTGATGCTAGGCATG
and
ZmTAS3jmut-F,
ccgcaataggaAATTGAGCTTTTCAGCCACCA.

### (2) Genetic Transformation of Arabidopsis

The plasmid AN101-ZmTAS3j and AN101-ZmTAS3jmut were transformed into Agrobacterium GV3101 respectively. Arabidopsis transgenic plants were obtained by inflorescence soaking. The process as follow:

1) Sow wild-type Arabidopsis seeds directly onto the surface of nutrient soil. Once the seedlings develop 4 to 5 true leaves, transplant them into a new culture box. Cultivate two plants per box and place them in a short sunlight incubator for vegetative growth. After the Arabidopsis seedlings have completed their vegetative stage, they are transferred to a long-day culture room for reproductive growth until the majority of the plants have bolted.

2) Inoculate the transformed Agrobacterium into 50 ml of medium containing the appropriate antibiotics (Kan and Rif at a concentration ratio of 1:1000) in YEP medium. Cultivate overnight at 28° C. with shaking at 200 rpm until the OD600 absorbance value reaches between 0.8 and 1.0.

3) Dip dye preparation: Prepare a dip dye solution (5% sucrose solution) in advance, then add surfactant (Silwet L-77) to the solution at a ratio of 1:10,000 to resuspend the bacterial solution.

4) Remove the bloomed flowers of wild-type Arabidopsis and soak the remaining inflorescences in the resuspended bacterial solution for 1 minute. After the transformation is complete, cover the Arabidopsis with a fresh-keeping bag and place it upside down to avoid light during overnight cultivation. The following day, culture the plants under normal lighting conditions. After one week, perform a second inflorescence dyeing.

5) Collect mature Arabidopsis seeds following genetic transformation. Mustard siliques should be dried in an oven at 37° C. and stored at 4° C.

6) Configure the ½ MS culture medium by adding the appropriate antibiotic (Kan). Wash the Arabidopsis seeds with 70% alcohol for 1 minute, followed by sterilization with 1% NaClO for 10 minutes. Subsequently, wash the seeds six times with sterile water. Spread the sterilized Arabidopsis seeds evenly on the ½ MS medium, using 60 to 100 seeds per dish. Allow the water on the surface of the seeds to air-dry, then cover and seal the dishes. Place them in a 4° C. refrigerator for vernalization treatment for 2 to 3 days. After this period, transfer the dishes to light conditions to screen for positive seeds. Positive plants, identified between 10 to 15 days, were those exhibiting green leaves, epicotyl elongation, and normal rooting. These positive plants were then transplanted into culture soil.

### (3) Identification of Transgenic Positive Plants

Transgenic lines screened using kanamycin (Kan) were collected and placed in 2.0 mL EP tubes, from which DNA was extracted through liquid nitrogen grinding. Specific PCR primers were designed based on the target sequence. Plants exhibiting target bands were identified as positive, utilizing the primers F: GACGTAAGGGATGACGCACA and R: ACGATCGGGGAAATTCGAGC.

(4) Phenotypic Identification of Arabidopsis thaliana Under Pb Stress

The 35S:ZmTAS3j (390_5D_390c) and 35S:ZmTAS3jmut (5D_390c) vectors were introduced into Arabidopsis (Col-0) via Agrobacterium-mediated transformation. Seeds were collected from positively homozygous lines of Arabidopsis in the T3 generation and germinated on ½ MS solid medium. After seven days, half of the seedlings from each transgenic line were transferred to ½ Murashige and Skoog (MS) solid media containing 200 mg/L Pb(NO₃)₂(Pb treatment), while the remaining seedling were transferred to ½ MS solid media without Pb(NO₃)₂(CK). Following 10 days of culture, Col-0 plants were served as negative controls. The Arabidopsis seedlings were assessed for phenotypic traits, including rosette diameter, plant height, root length, number of lateral roots, and total root length. Measurements of rosette diameter, plant height, and root length were conducted using a ruler. The root systems were scanned with an Epson Expression 10000XL scanner, and the images were analyzed using WinRHIZO software to quantify the number of lateral roots and total root length. The phenotypic trait data for Arabidopsis thaliana were statistically analyzed using IBM SPSS Statistics version 19.

### 4.2 Results and Analysis

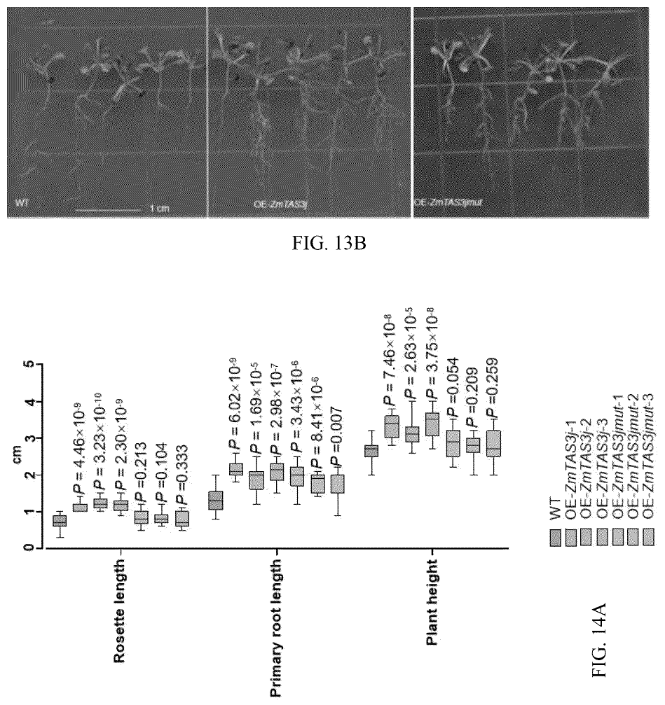

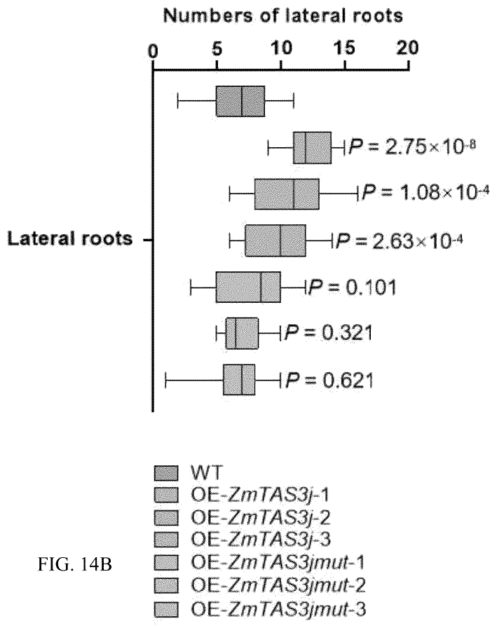

To investigate the role of ZmTAS3j in plant tolerance to Pb stress, we cultivated Col-0, ZmTAS3j-OE, and ZmTAS3jmut-OE lines under both control conditions (½ MS medium) and Pb stress conditions (½ MS medium containing 200 mg/L Pb(NO₃)₂). The ZmTAS3j-OE lines exhibited more robust root systems compared to the ZmTAS3jmut-OE and Col-0 plants under both control and Pb treatment conditions (FIGS. 13A and B). Furthermore, under Pb stress, the ZmTAS3j-OE lines demonstrated significantly increased rosette size, plant height, primary root length, and lateral root number compared to Col-0 (FIGS. 14A and B). In contrast, no significant differences were observed between the ZmTAS3jmut-OE and Col-0 plants for a forementioned traits, except for primary root length, which was notably greater in the ZmTAS3jmut-OE lines than in Col-0 plants (FIGS. 14A and B). These findings indicate that ZmTAS3j acts as a positive regulator of Pb tolerance in Arabidopsis.

### 5. Overexpression of ZmTAS3j Promoted Maize Root Growth Under Pb Stress.

### 5.1 Materials and Methods

### 5.1.1 Materials

Maize inbred line KN5585, overexpressed strain (OE) obtained with KN5585(WT) as background material

### 5.1.2 Methods

### (1) Construction of Overexpression Vector.

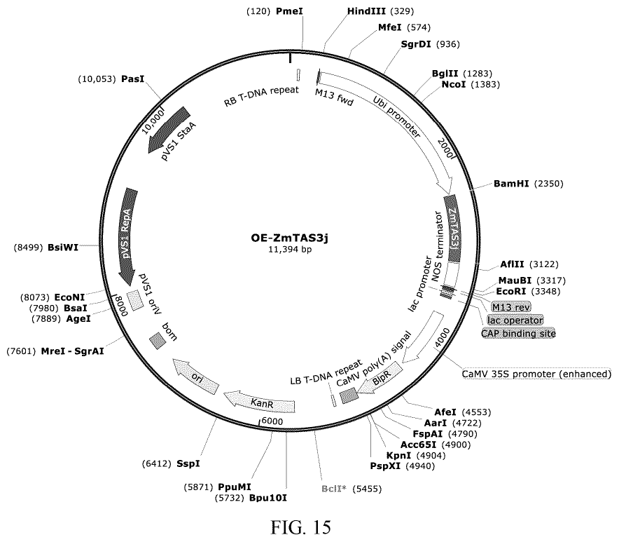

(1) The sequences of ZmTAS3j (718 bp) were cloned into the pCUB vector under the control of the ubiquitin (Ubi) promoter (FIG. 15). The ZmTAS3j-overexpressing (OE) lines was generated under the backgrounds of the maize line KN5585, respectively, through Agrobacterium-mediated transformation. The primer sequence: OE-ZmTAS3j-F: gcaggtcgactctagaggatccGCGGTTTCGTTCTCCTTCCTGC; OE-ZmTAS3j-R: tcgagctcggtacccggggatccGCAGCAGCTTATTCCATCGAGGC.

### (2) Positive Identification of Transgenic Lines of Maize

In this study, the PCR detection method for cut endosperm was employed to identify positive seeds from each transgenic line. Two pairs of primers were designed for this purpose. The first primers based on the sequence of the marker gene Bar (F: CCATCGTCAACCACTACATCGAGACA and R: CTTCAGCAGGTGGGTGTAGAGCGT). To further minimize the occurrence of false positives, a section of the Ubi promoter sequence was utilized as the forward primer (F: TTGTCGATGCTCACCCTGTTG), while a sequence from the target gene ZmTAS3j was used as the reverse primer (R: CTCATCATGCCAAGCGGACA). Through PCR identification using these two pairs of primers, positive overexpression seeds were successfully screened for subsequent experiments.

### (3) RNA Extraction and RT-qPCR Assays

Total RNA was extracted from N. benthamiana, Arabidopsis, and maize using the HiPure Plant RNA Mini Kit (Magen, R4165-03, Guangzhou, China). The PrimeScript™_RT reagent kit with gDNA Eraser (Takara, Dalian, China) was employed to convert mRNAs into cDNA. RT-qPCR was performed using the ABI7500 real-time PCR system and SYBR (NovoStart SYBR qPCR SuperMix Plus) to quantify gene expression levels. sRNAs were extracted using the HiPure Universal miRNA Kit (Magen, Guangzhou, China). The Mir-X miRNA first-strand synthesis kit (Clontech, Mountain View, CA, USA) was utilized for the reverse transcription, followed by RT-qPCR to quantify their expression levels of sRNAs. The Actin primer sequences were as follows: F: TGG AATCC ACG AG AC AACCTA, R: TTCTG TG AACG ATTCCTGG AC; and the U6 primer sequences were: F: CTCGCTTCGGCAGCACA R: AACGCTTCACGAATTTGCGT.

### (4) Construction and Phenotyping of ZmTAS3j-Overexpression Lines in Maize

Seeds collected from both transgenic and wild-type lines were sterilized using a 2% (w/v) NaClO solution for 15 minutes and subsequently rinsed three times with distilled water. The sterilized seeds were then soaked overnight in a saturated solution of CaSO₄·H₂O₂, followed by five additional rinses with distilled water. Afterward, the seeds were sown on wet filter paper to initiate germination. Once germinated, the seeds were planted in quartz sand and grown in a greenhouse under a light/dark cycle of 14 hours at 28° C. and 10 hours at 22° C., maintaining a relative humidity of 70%. At the two-leaf stage, the seedlings were transferred to a modified Hoagland solution, which was supplemented with 1 mM Pb(NO₃)₂ for the Pb treatment. The pH of the solution was maintained at 4.5±0.1 by titration with 0.1 M HCl or NaOH as necessary. After seven days of culture, the seedlings were evaluated for root dry weight (RDW), total root length (TRL), primary root length (PRL), and lead (Pb) concentrations in both shoots and roots. TRL and PRL measurements were conducted using an Epson Expression 10000XL scanner in conjunction with WinRHIZO software.

### 5.2 Results and Analysis

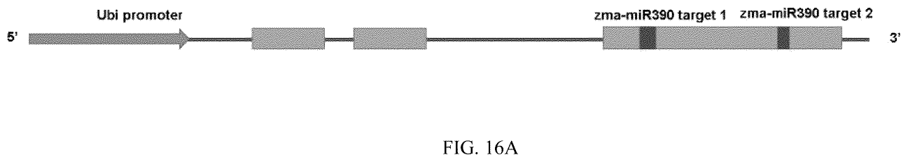

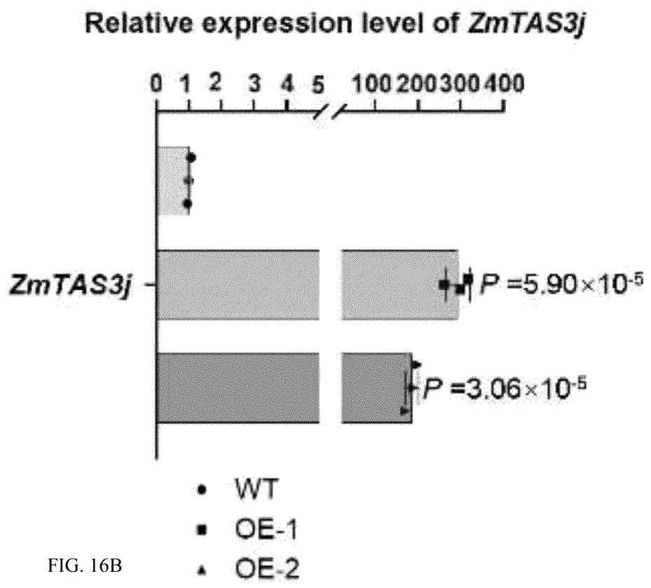

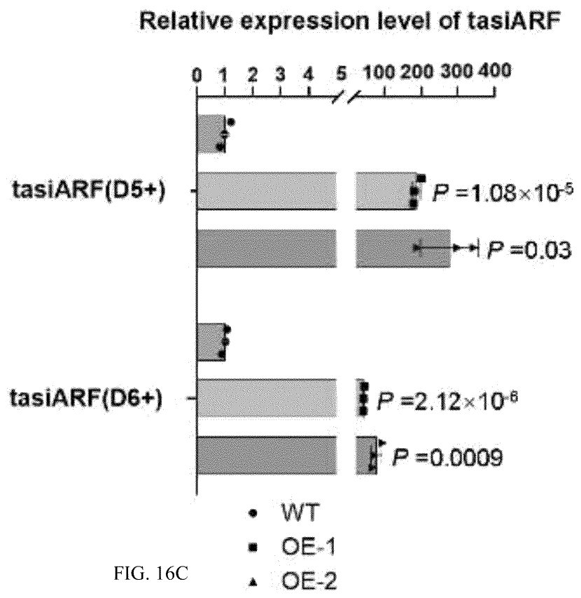

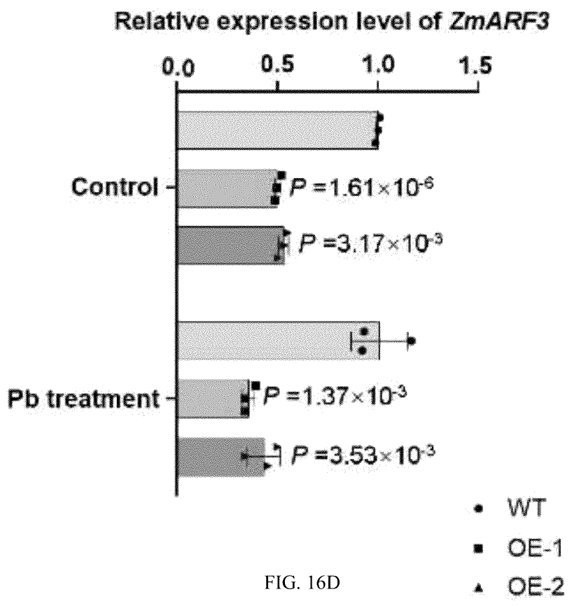

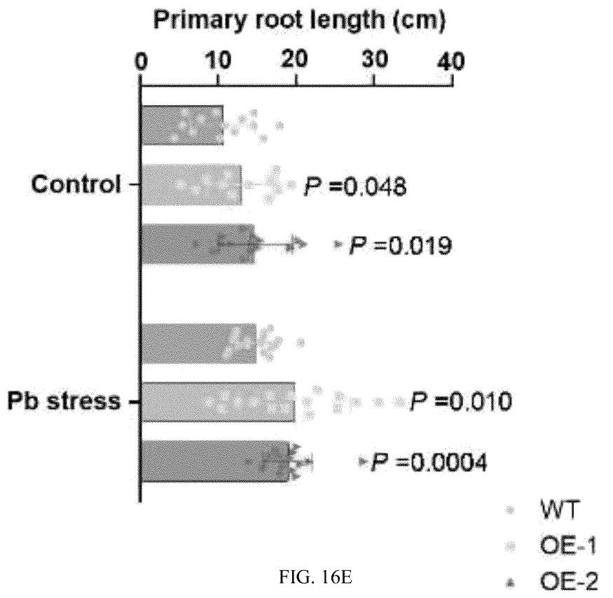

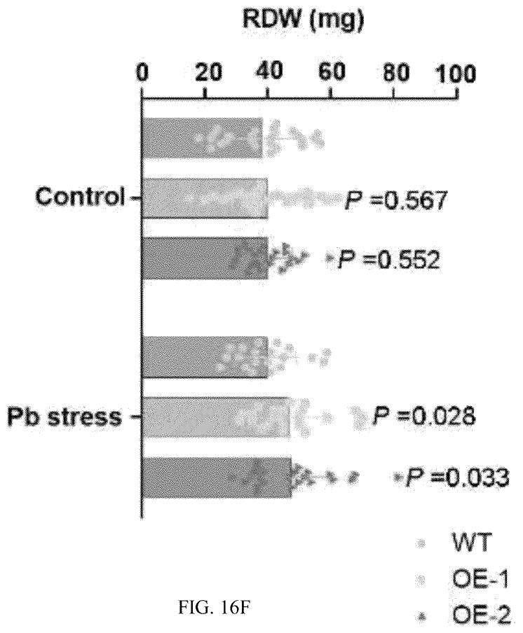

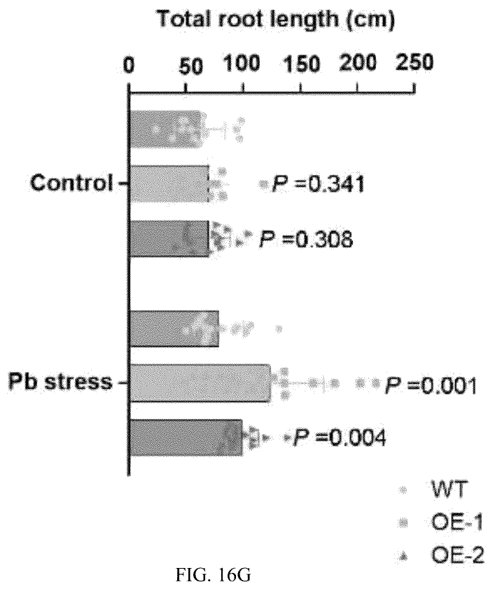

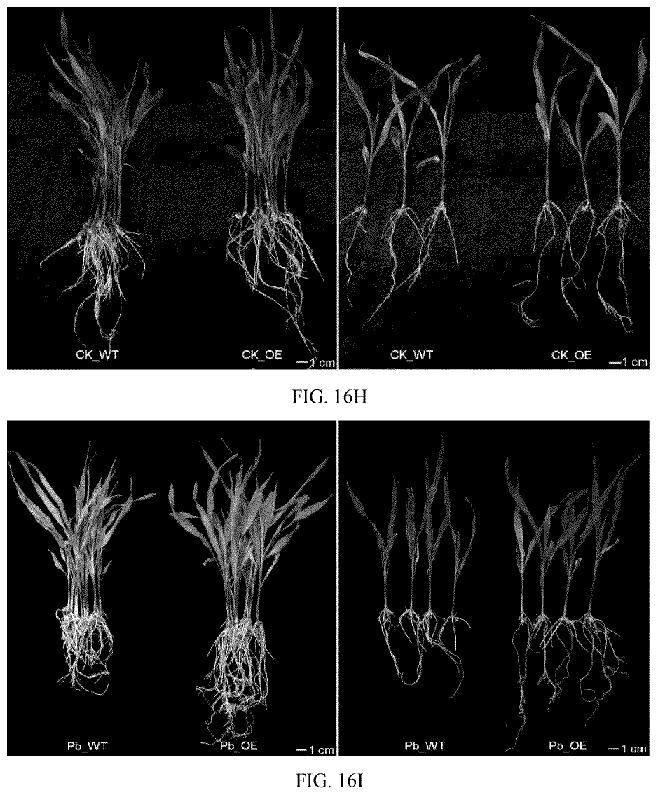

To further investigate the role of ZmTAS3j in maize tolerance to Pb stress, we generated two independents maize ZmTAS3j overexpression (OE) lines (FIG. 16A). The expression levels of ZmTAS3j and tasiARFs (D5(+) and D6(+)) were found to be higher in the OE lines (OE1 and OE2) compared to those in the wild-type (WT) plants (FIGS. 16B and C). Additionally, ZmARF3 exhibited downregulated expression in the OE lines relative to WT (FIG. 16D), confirming that ZmARF3 is targeted and cleaved by the overexpressed ZmTAS3j-derived tasiARFs in maize.

The OE lines underwent phenotypic evaluation under Pb stress. Overexpression of ZmTAS3j resulted in significant increases in primary root length (PRL), with percentage increases ranging from 30.90% to 38.35% under Pb treatment and from 27.28% to 33.13% under control (CK, without Pb) conditions, compared to wild-type (WT) plants (FIG. 16 E, H, and I). Notably, under Pb stress, the root dry weight (RDW) and total root length (TRL) were significantly enhanced in the OE lines by 18.47% to 19.74% and 24.88% to 56.02%, respectively, compared to WT plants (FIGS. 16F, G, H, and I). However, no significant differences in RDW and TRL were observed between the OE lines and WT under CK conditions (FIGS. 16F and G). Collectively, these results suggest that overexpression of ZmTAS3j in maize promotes the growth of primary roots, thereby positively influencing the length and weight of the entire root system under Pb stress.

### 6. Overexpressed ZmTAS3j Reduces Cell Wall Thickness and Pb Aggregate Accumulation in Maize Root Cells

### 6.1 Materials and Methods

### 6.1.1 Materials

The materials were KN5585(WT) and OE-ZmTAS3j lines.

### 6.1.2 Methods

The ultramicrolocalization of lead (Pb) distribution in root cells and the determination of Pb content were conducted using transmission electron microscopy (TEM) and energy-dispersive spectroscopy (EDS). Wild-type (WT) and overexpressing (OE) roots were subjected to 7 days of Pb stress and underwent a series of preparatory steps, including fixation with formaldehyde-acetic acid-alcohol (FAA), elution, osmium tetroxide fixation, cleaning, replacement, infiltration, embedding, polymerization, and ultrasonic thin sectioning. These sections were subsequently stained and examined microscopically. TEM was utilized to observe and photograph the ultrastructure of the ultrathin sections, while EDS facilitated the elemental analysis of black dots or aggregates present. Following seven days of 1 mM Pb(NO₃)₂ treatment, maize seedlings were immersed in 20 mM disodium ethylenediamine tetra-acetic acid (Na₂-EDTA) and rinsed three times with distilled water. After drying the shoots and roots to a constant weight, samples from each strain were divided into three replicates for the determination of Pb content in both the shoots and roots. This procedure is consistent with the Pb content determination method outlined in section 1.1.2.

### 6.2 Results and Analysis

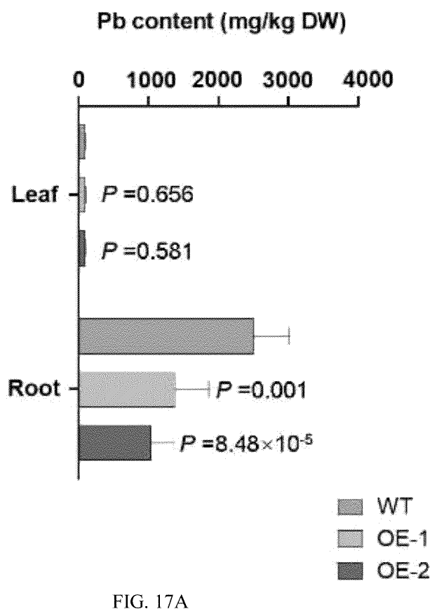

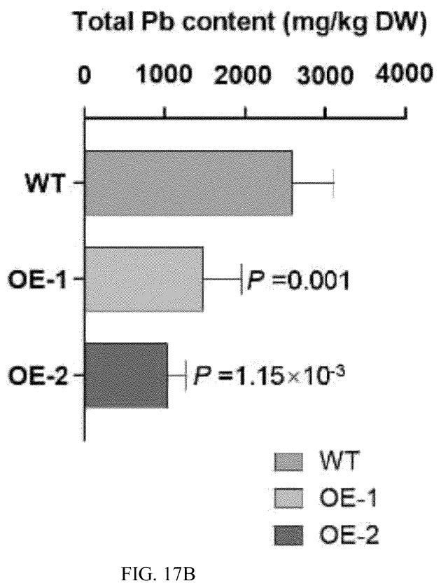

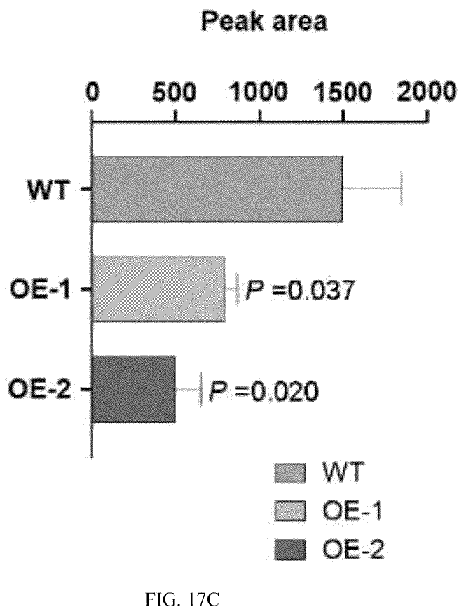

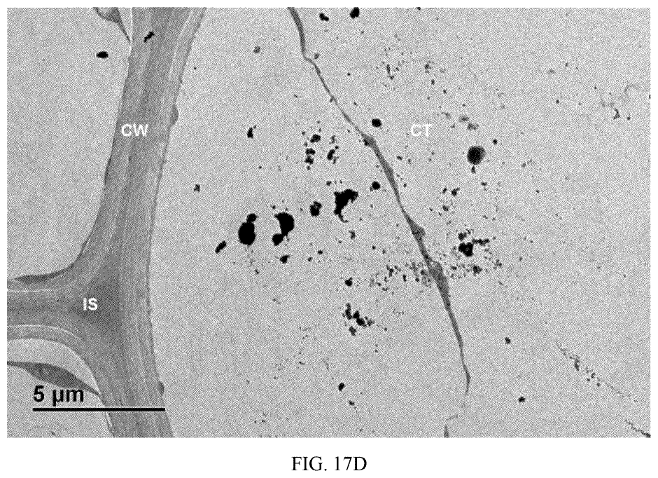

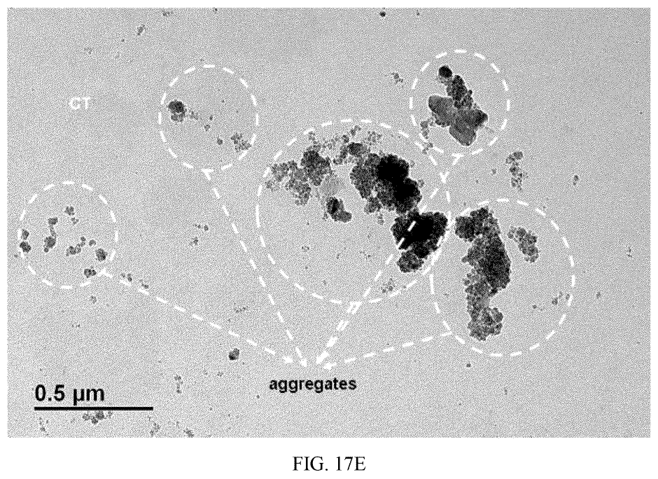

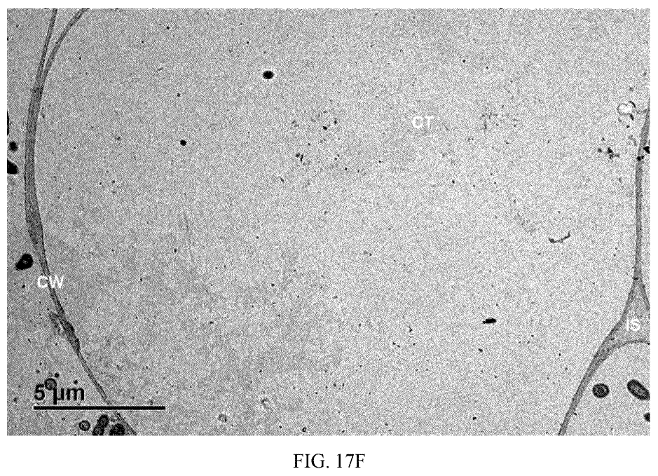

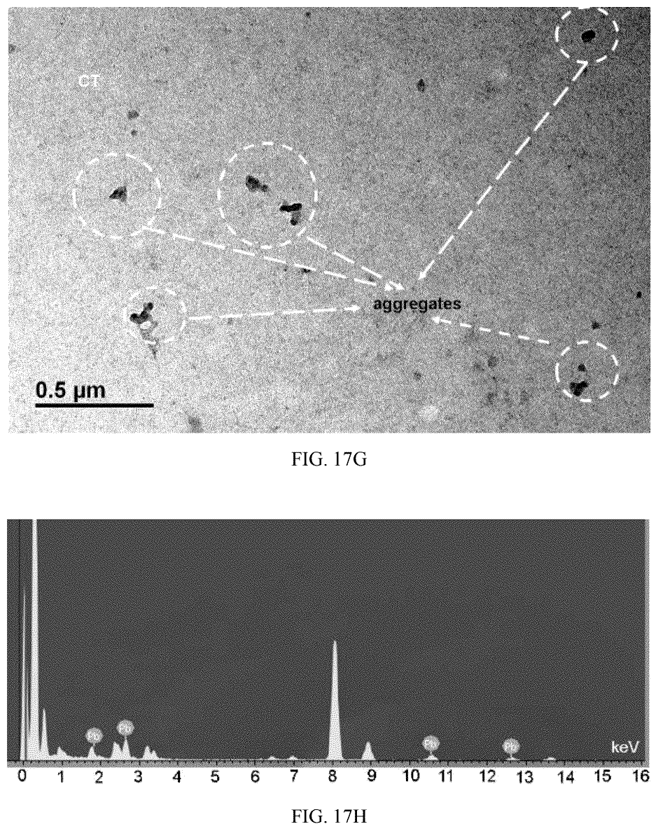

To investigate the effects of ZmTAS3j on Pb accumulation, we cultured ZmTAS3j-OE and WT maize lines under 1 mM Pb(NO3)2 treatment for seven days. The roots and shoots were analyzed separately for Pb content using inductively coupled plasma mass spectrometry. In the shoots, no significant difference in Pb content was observed between the OE and WT lines (FIG. 17A). In the roots, the Pb content in the OE lines was measured at 1574.52 mg/kg DW and 1032.68 mg/kg DW, which were significantly (P<0.001) lower than that of the WT line (2339.54 mg/kg DW) (FIG. 17A). Consistent with these findings, the total Pb content in the entire seedlings was significantly (P<0.001) lower in the OE lines compared to the WT line (FIG. 17B).

Transmission Electron Microscopy-Energy Dispersive Spectrometer (TEM-EDS) analysis revealed that Pb ions and their aggregates penetrated the cell membrane and entered the cytoplasm (FIG. 17 D, E, F, and G). Notably, the cell walls (CWs) of the wild-type (WT) roots exhibited thickening in response to Pb treatment, resulting in increased concentrations of Pb ions and their aggregates within the root cells, in contrast to the unchanged CW thickness observed in the overexpressing (OE) lines (FIGS. 17D and F). Furthermore, the internalized Pb aggregates were directly absorbed by the maize seedling roots, with ten distinct Pb compounds localized in the root cells of both the OE and WT lines. Among these compounds, four displayed pronounced peaks (FIGS. 17H and I). Importantly, the total area of these peaks, indicative of Pb aggregates, was significantly smaller (P<0.05) in the OE line compared to the WT (FIGS. 17C, H, and I). Collectively, these findings suggest that ZmTAS3j negatively regulates both cell wall thickness and Pb aggregate accumulation in maize root cells.

### 7. Overexpressed ZmTAS3j Enhances IAA Level and Scavenging Capacity of ROS in Maize

### 7.1 Materials and Methods

### 7.1.1 Materials

The roots of the ZmTAS3j-OE (OE-1, OE-2) and WT plants were collected after Pb treatment for 0, 24, 48, and 72 h.

### 7.1.2 Methods

### (1) Determination of IAA Level

The concentration of IAA in the samples was determined using a double-antibody sandwich enzyme-linked immunosorbent assay (ELISA) detection kit. Specific absorption was measured at a wavelength of 450 nm using a microplate reader, and the IAA concentration in the samples was calculated based on the standard curve.

### (2) Determination of Antioxidant System Activity and DAB Staining

The activities of SOD, POD, CAT, and H₂O₂ were assayed for the above root samples using the “SOD detection Kit, Solarbio BC0175”, “POD detection Kit, Solarbio BC0095”, “CAT detection Kit, Solarbio BC0205”, and “H₂O₂ detection Kit, Solarbio BC3559”, respectively. IAA levels were determined using an enzyme-linked immunosorbent assay (ELISA) kit (A600724-0100).

Meanwhile, after 72 h of Pb treatment, the leaves of the OE (OE-1, OE-2) and wild-type plants were collected to detect H₂O₂ presence and distribution using the DAB staining method. Briefly, the leaves with Pb treatment were incubated in 1% DAB solution for 24 h at room temperature. The leaves were then boiled in bleaching solutions (75% ethanol) to remove chlorophyll, and images of the leaves were taken using an Epson Expression 10000XL scanner.

### 7.2 Results and Analysis

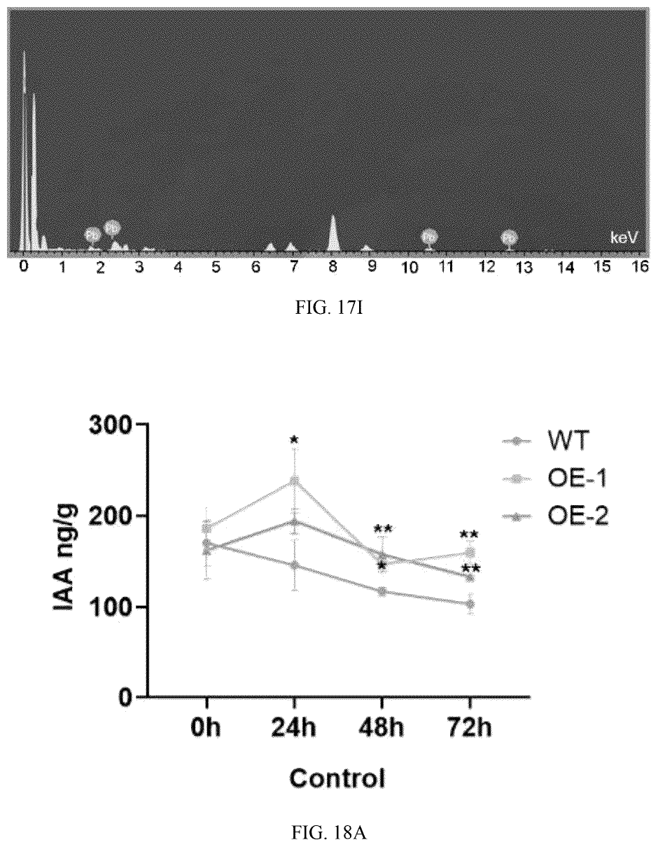

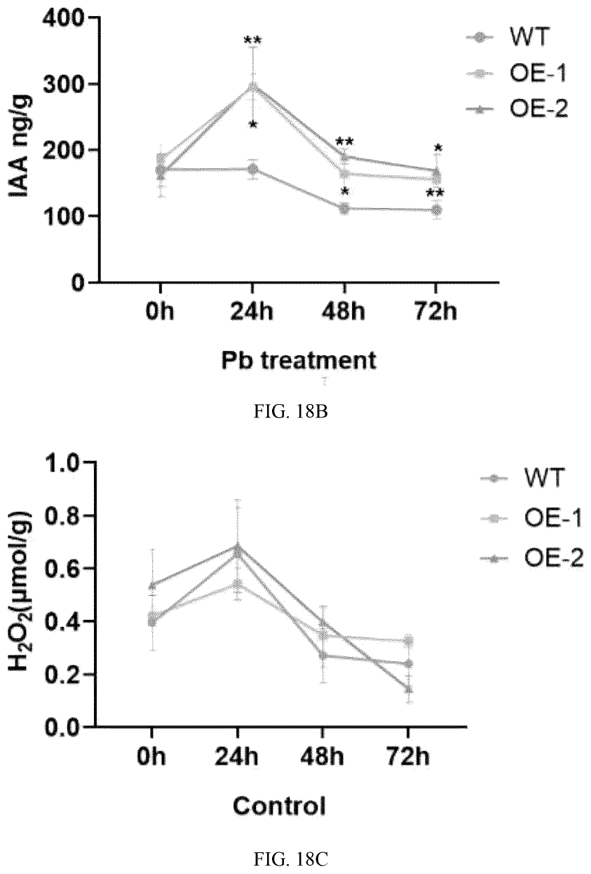

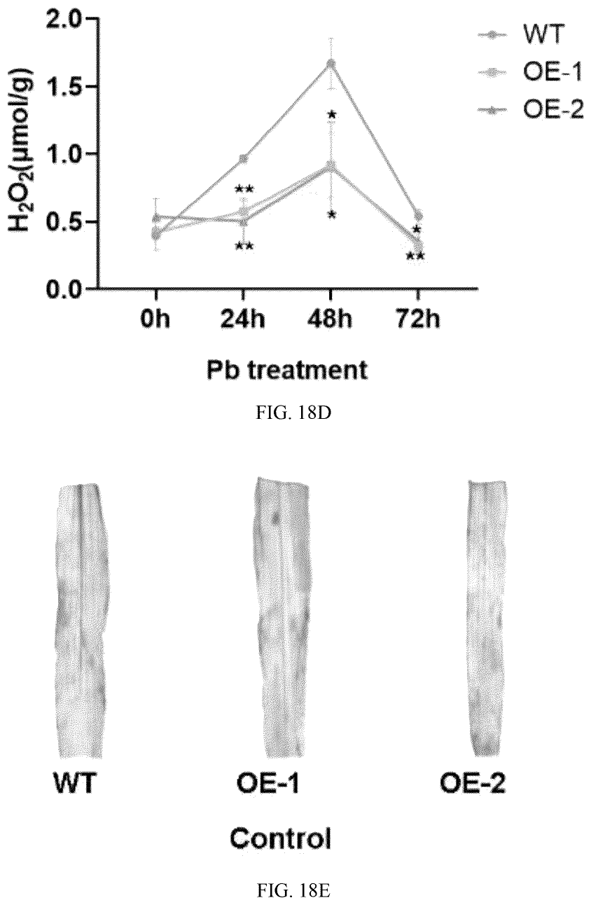

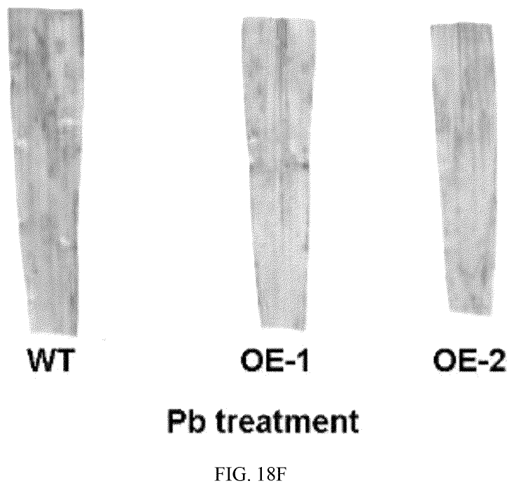

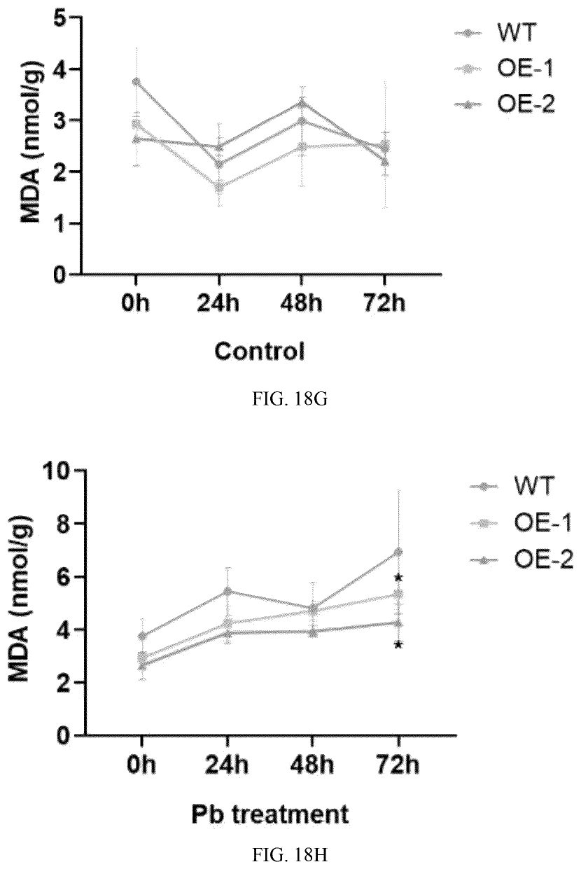

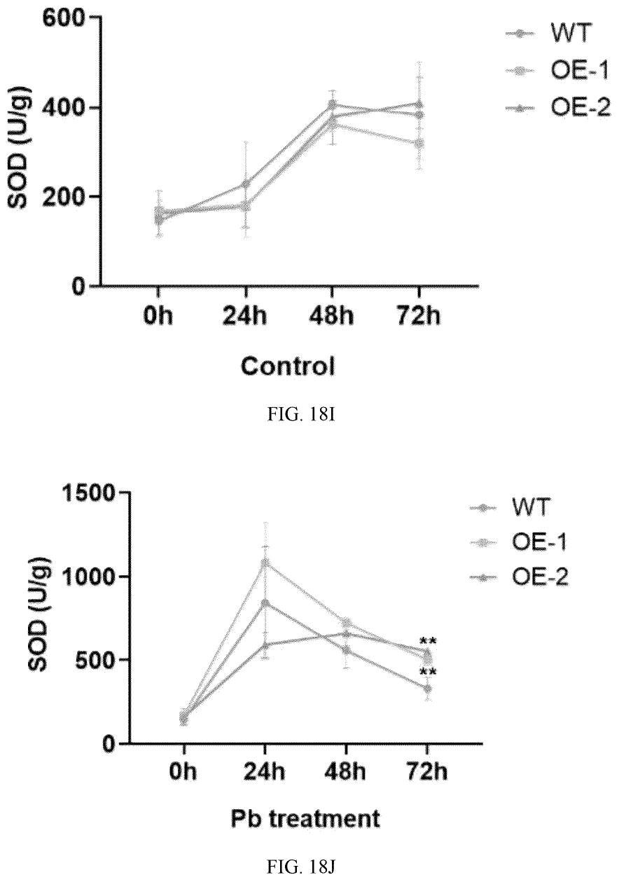

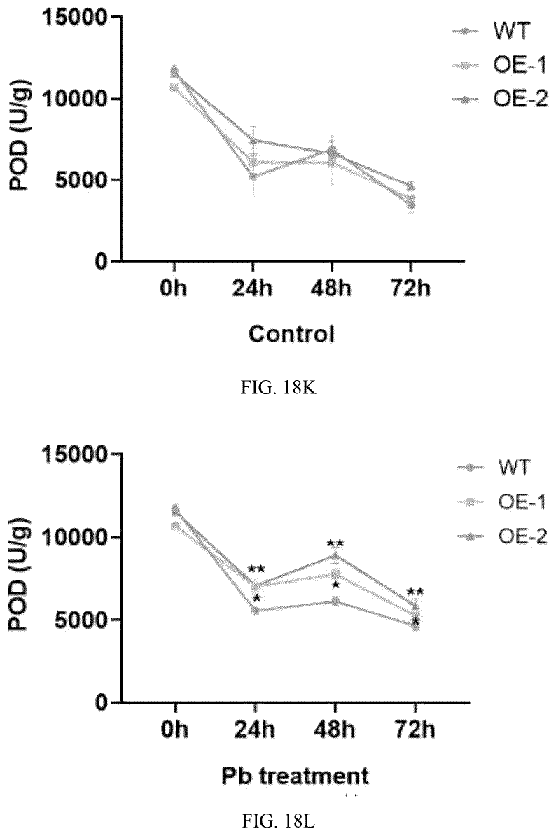

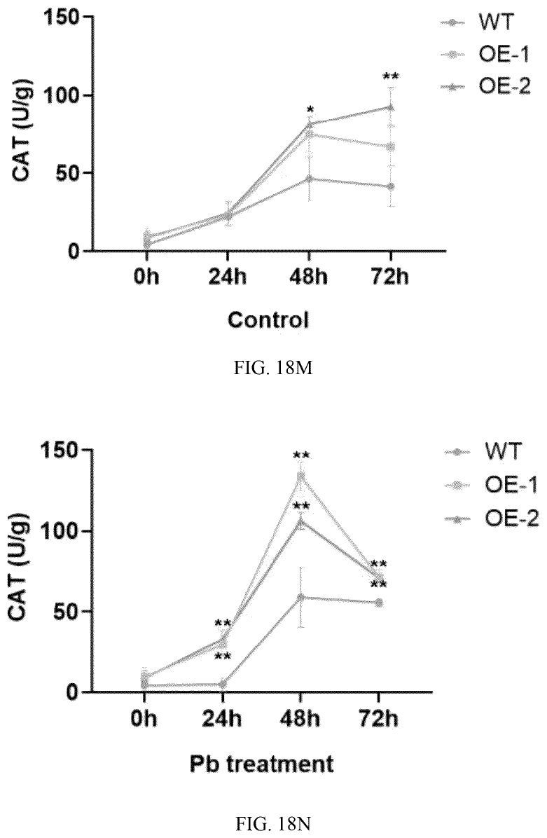

To investigate whether ZmTAS3j-mediated Pb tolerance is associated with the positive role of ZmTAS3j in indole acetic acid (IAA) accumulation, we measured IAA content in the roots of the overexpressing (OE) and wild type (WT) lines at different stages of control (CK) and Pb treatment. Compared to CK, Pb treatment resulted in a consistent increase in IAA content at each stage in the OE roots (FIGS. 18A and B). Following Pb treatment, IAA content peaked at 24 hours and subsequently declined to lower levels at 48 and 72 hours across all lines (FIG. 18B). Notably, the OE lines exhibited higher IAA content than the WT lines at every stage of Pb stress (FIG. 18B).

To further investigate the regulatory role of ZmTAS3j in lead (Pb) stress during the seedling stage of maize, this study assessed the levels of hydrogen peroxide (H₂O₂) and malondialdehyde (MDA), as well as the activity of antioxidant system enzymes in maize seedlings. Initially, H₂O₂ was measured in the leaves of both overexpressing (OE) and wild-type (WT) plants under control conditions and Pb stress treatment over a period of 7 days, as well as in the roots after treatment durations ranging from 0 to 72 hours. Under control conditions, no significant differences were observed in the H₂O₂ content of the roots and leaves between OE and WT plants (FIGS. 18C and 17E). Conversely, under Pb stress conditions, H₂O₂ levels in the leaves and roots of all strains were lower than those observed under control conditions. Although there was an increase in H₂O₂ levels, the amounts in the leaves and roots of the OE line were significantly lower than those in the WT plants (FIGS. 18D and 17F). Concurrently, measurements of MDA indicated no significant differences between the wild-type and transgenic lines under control conditions (FIG. 18G). During Pb stress treatment over the 0 to 72-hour period, MDA content in the OE lines was consistently lower than in the WT, with a significant difference observed between the two OE lines and the WT at the 72-hour mark (FIG. 18H).

In summary, the overexpression of ZmTAS3j can reduce the levels of H₂O₂ and MDA, thereby mitigating damage to plants and enhancing their tolerance to Pb. Furthermore, the activities of the antioxidant enzymes SOD, POD, and CAT were evaluated. Under control conditions, significant differences were observed in the activities of the three antioxidant enzymes in the root systems of overexpressing (OE) and wild-type (WT) plants, with the exception of CAT at 48 and 72 hours. However, no significant differences were noted in other growth processes (FIGS. 18I, K, and M). After 72 hours of Pb treatment, SOD activity in the roots of the OE strain was significantly higher than that observed in the WT (P<0.05; FIG. 18J). Throughout the duration of Pb stress treatment from 0 to 72 hours, both POD and CAT activities in the roots of the OE strain were significantly elevated compared to the WT strain (P<0.05; FIGS. 18L and N).

Overall, the overexpression of ZmTAS3j not only increases the IAA levels in the roots of maize seedlings but also promotes root growth under lead (Pb) stress. Furthermore, the enhanced activity of reactive oxygen species (ROS) scavenging enzymes facilitates the removal of ROS within the plant, thereby mitigating ROS-induced damage and improving the tolerance of maize seedlings to Pb stress.

### 8. Regulatory Network of ZmTAS3j Implies Importance of SAURs in Pb Tolerance

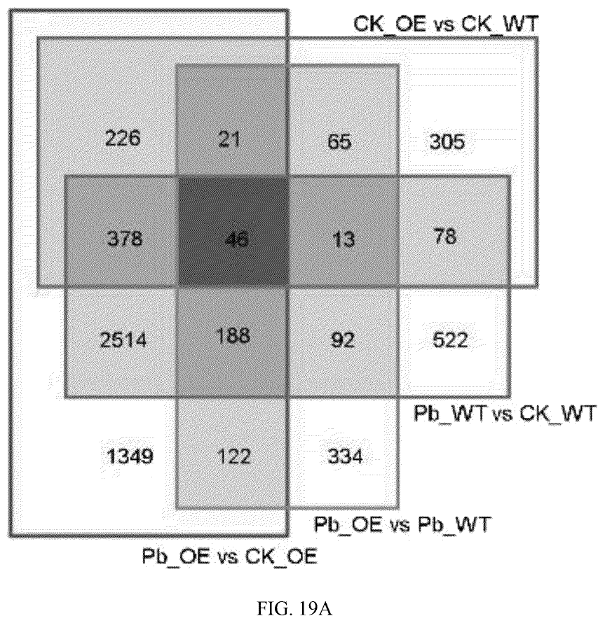

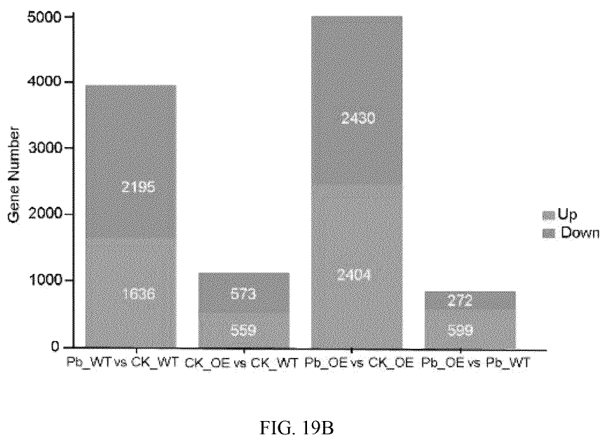

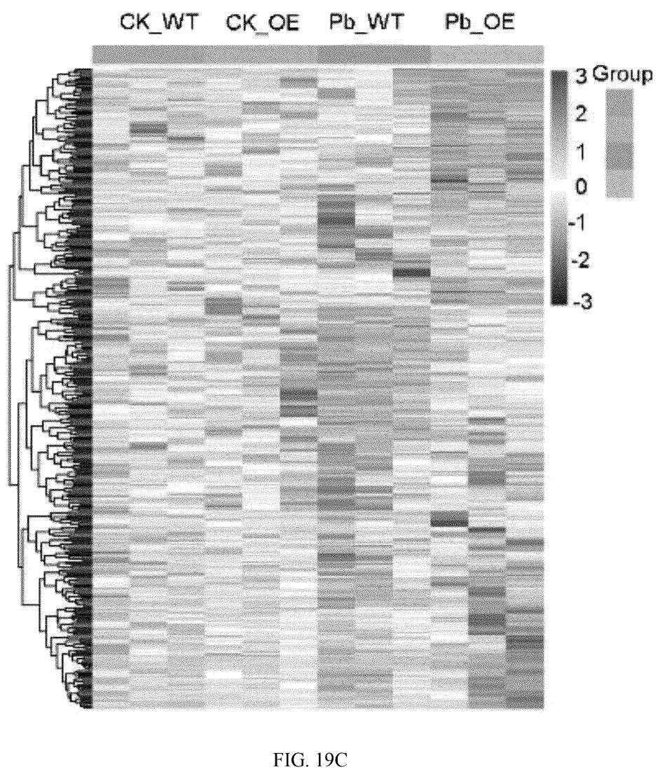

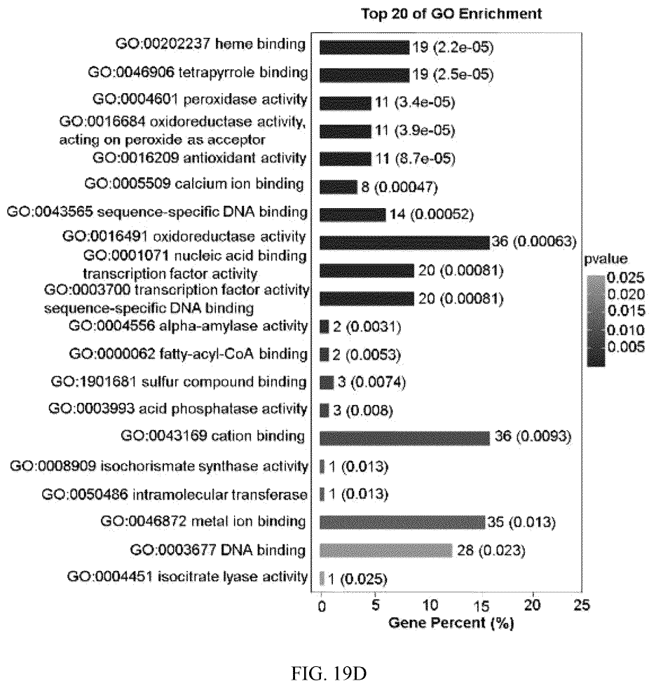

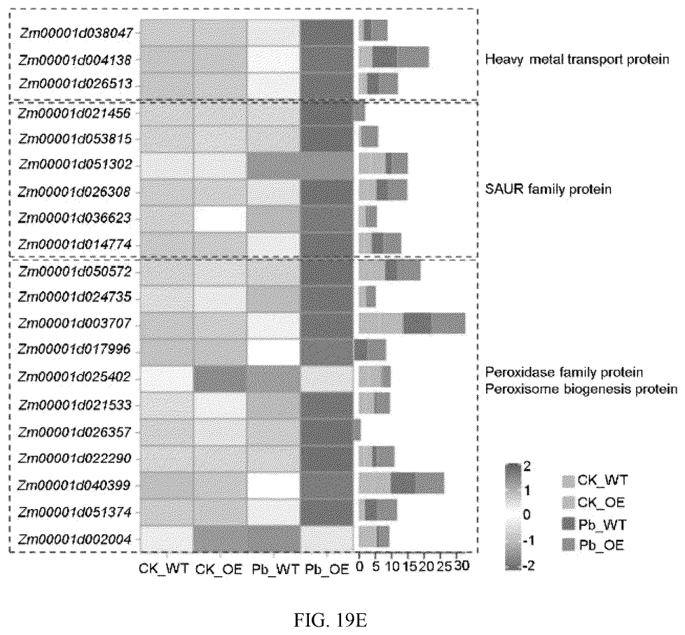

To investigate the regulatory network of ZmTAS3j associated with Pb tolerance in maize, transcriptome analyses were conducted to identify differentially expressed genes (DEGs) in the ZmTAS3j-OE maize line compared to the wild type (WT) under both control (CK) and Pb stress conditions (FIG. 19A). Under CK conditions, a total of 1132 DEGs were specifically expressed between the OE and WT lines (CK_OE vs. CK_WT), with 559 upregulated and 573 downregulated (FIG. 19B). Following Pb treatment, the OE line exhibited 599 upregulated and 272 downregulated DEGs in comparison to WT (Pb_OE vs. Pb_WT) (FIG. 19B). Between the CK and Pb treatment conditions, 3831 DEGs were identified in WT, comprising 1636 upregulated and 2195 downregulated DEGs, while 4834 DEGs were identified in the OE line, including 2404 upregulated and 2430 downregulated DEGs (FIG. 19B). Notably, 334 DEGs in the OE line were specifically expressed under Pb stress (FIG. 19 A and C), which were primarily enriched in biological processes such as response to peroxidase, oxidoreductase and antioxidant activity, as well as cation and metal ion binding (FIG. 19D). This finding is further supported by observations of an increased scavenging capacity for reactive oxygen species (ROS) in the ZmTAS3j-OE lines. Remarkably, the expression of several small auxin-upregulated RNA (SA UR) genes (Zm00001d014774, Zm00001d021456, Zm00001d036623, Zm00001d026308, Zm00001d051302, and Zm00001d053815), peroxidase family proteins (Zm00001d003707, Zm00001d017996, Zm00001d025402, Zm00001d024735, Zm00001d002004, Zm00001d022290, Zm00001d021533, Zm00001d026357, Zm00001d040399, and Zm00001d050572), a peroxisome biogenesis protein (Zm00001d051374), heavy metal transport/detoxification superfamily proteins (Zm00001d038047 and Zm00001d026513), and heavy metal-associated isoprenylated plant protein 27 (Zm00001d004138) were significantly upregulated in the OE line specifically under Pb stress (FIG. 19E). Our previous study indicated that SAUR genes were associated with IAA biosynthesis in maize, which explained for the enhanced IAA content and promoted root growth in the ZmTAS3j-OE lines.

What is claimed is:

1. Maize ZmTAS3j Gene for use in mediating lead stress tolerance.

2. The Maize ZmTAS3j Gene of claim 1, wherein the nucleotide sequence of ZmTAS3j is identified by SEQ ID NO:1.

3. A method for improving plant tolerance to lead stress, comprising overexpressing ZmTAS3j in plant.

4. The method of claim 3, wherein the step of overexpressing ZmTAS3j in plant comprising overexpressing ZmTAS3j in maize, which comprises: with Ubi as the promoter, nos as the terminator, bar as the selective marker gene, designing amplification primers according to sequence of ZmTAS3j, and amplifying the ZmTAS3j; and
constructing overexpression vector of OE-ZmTAS3j by homologous recombination using BamHI as the restriction site.

5. The method of claim 4, wherein primer used in the homologous recombination is OE-ZmTAS3j-F identified by SEQ ID NO:6, or OE-ZmTAS3j-R identified by SEQ ID NO:7.

6. A method of ZmTAS3j-overexpression in Arabidopsis thaliana, comprising steps of:
(1) extracting total RNA of B73 inbred lines and reverse-transcribing the same into cDNA, amplifying the sequences of ZmTAS3j, and constructing the sequences of ZmTAS3j into pRI101-AN expression vector with EcoRI as the restriction site, wherein the pRI101-AN expression vector was named AN101-ZmTAS3j;
(2) mutating 5′ target of miR390 in the sequence of ZmTAS3j by mismatch PCR amplification, and destroying the 5′ target of miR390, constructing the mutant sequence of ZmTAS3j into pRI101-AN expression vector with EcoRI as the restriction site, wherein the pRI101-AN expression vector was named AN101-ZmTAS3jmut;
(3) transforming plasmid AN101-ZmTAS3j and AN101-ZmTAS3jmut into Agrobacterium GV3101 respectively, and obtaining Arabidopsis transgenic plants by inflorescence soaking.

7. The method of claim 6, wherein homologous recombination primer used in the step (1) comprises AN101-ZmTAS3j-F identified by SEQ ID NO: 2, and AN101-ZmTAS3j-R identified by SEQ ID NO: 3.

8. The method of claim 6, wherein homologous recombination primer used in the step (2) comprises ZmTAS3jmut-FL-R identified by SEQ ID NO: 4, and ZmTAS3jmut-FL-F identified by SEQ ID NO: 5.
